(19) United States
(12) Patent Application Publication
(10) Pub. No.: US 2017/0335411 A1
(43) Pub. Date: Nov. 23, 2017

(54) CORRECTING CROSSTALK IN BIOLOGICAL SYSTEMS

(71) Applicant: Massachusetts Institute of Technology, Cambridge, MA (US)

(72) Inventors: Timothy Kuan-Ta Lu, Cambridge, MA (US); Isaak Elis Mueller, Cambridge, MA (US); Jacob Rosenblum Rubens, Cambridge, MA (US)

(73) Assignee: Massachusetts Institute of Technology, Cambridge, MA (US)

(21) Appl. No.: 15/521,917

(22) PCT Filed: Oct. 27, 2015
(86) PCT No.: PCT/US15/57478

§ 371 Date: Apr. 26, 2017

Related U.S. Application Data
(60) Provisional application No. 62/069,132, filed on Oct. 27, 2014.

(51) Int. Cl. C12Q 1/68; C12N 15/70; C12N 9/50; G06N 3/00; C07K 14/245

(52) U.S. Cl. CPC C12Q 1/6897; G06N 3/002; C12Y 304/22044; C07K 14/245; C12N 9/506; C12N 15/70; C07K 2319/50

(57) ABSTRACT

Aspects of the present disclosure are directed to biosensing circuits that correct crosstalk.

### RELATED APPLICATIONS

This application claims the benefit under 35 U.S.C. §119(e) of U.S. provisional application No. 62/069,132, filed Oct. 27, 2014, which is incorporated by reference herein in its entirety.

### FEDERALLY SPONSORED RESEARCH

This invention was made with Government support under Grant No. CCF-1124247 awarded by the National Science Foundation. The Government has certain rights in the invention.

### FIELD OF THE INVENTION

Embodiments of the present disclosure relate to the field of biosynthetic engineering.

### BACKGROUND OF THE INVENTION

An overarching goal of synthetic biology is to engineer living organisms to execute tasks in defined environments and contexts. Such engineering is aided by abstraction into modules: sensing modules, which take stock of the environment; computing units, which integrate input signals and decide upon the course of action given previous information; and actuator models, which implement the tasks. Many biosensing circuits have been engineered to operate digitally, which is advantageous when signals do not crosstalk and binary information or a single threshold is sufficient to make a decision. However, digital biosensors are not useful when analog information, such as the sum or ratio of signals, is necessary for decision-making when graded responses to a signal are necessary. After sensing signals, signal integration in computing units is complicated by the fact that synthetic systems are fundamentally coupled to the cell's endogenous metabolic and gene networks. Consequently, crosstalk can arise between sensors and/or endogenous networks, affecting the faith of signal integration.

### SUMMARY OF THE INVENTION

A major challenge in understanding and designing cellular signaling networks is the presence of crosstalk between pathways. The predominant paradigm in synthetic gene network design is to minimize crosstalk at the input and process signals independently of each other, and then integrate information with Boolean logic. However, insulating signal processing networks from each other within individual cells is challenging, especially as gene networks increase in size. The present disclosure shows that analog gene circuits can function despite signal integration with other gene networks by designing circuits that compensate for crosstalk. This general principle is demonstrated by engineering biosensing circuits for reactive oxygen species (ROS) in Escherichia coli. Biosensing circuits were designed to maximize their analog computational capacity based on a novel metric, called utility. The initial ROS-sensing circuits exhibited unwanted crosstalk between two different ROS inputs (e.g., hydrogen peroxide and paraquat). This crosstalk was reduced via synthetic gene circuits that intentionally introduced counter-crosstalk, thus resulting in circuits capable of discriminating between the analog concentration of different ROS species. Engineered bacteria containing the biosensing circuits were able to differentiate between dendritic cells derived from normal mice versus those from a mouse model of inflammatory bowel disease. Correcting natural crosstalk with artificial crosstalk can be generalized to design genetic sensing networks with optimized analog behaviors.

Some embodiments of the present disclosure provide biosensing circuits comprising (a) a first promoter responsive to a first input signal and operably linked to a nucleic acid encoding a first output molecule; and (b) a second promoter responsive to the first input signal and operably linked to a nucleic acid encoding a copy of the first output molecule, wherein the response of the second promoter to the first input signal is opposite the response of the first promoter to the first input signal such that the first input signal does not affect relative production of the first output molecule.

In some embodiments, the first promoter is responsive to a first input signal and a second input signal.

In some embodiments, (a) and (b) are on the same vector.

In some embodiments, production of the first output molecule of (a) is decrease as a result of the first promoter responding to the first input signal.

In some embodiments, production of the copy of the first output molecule of (b) is increased as a result of the second promoter responding to the first input signal.

In some embodiments, production of the first output molecule of (a) is increased as a result of the first promoter responding to the second input signal.

In some embodiments, biosensing circuits further comprise a third promoter responsive to the first input signal and operably linked to a nucleic acid encoding a second output molecule that is different from the first output molecule.

In some embodiments, biosensing circuits further comprise a fourth promoter operably linked to a nucleic acid encoding a first biomolecule that binds to and regulates the first promoter and is responsive to the second input signal.

In some embodiments, activity of the first biomolecule is induced by the second input signal.

In some embodiments, biosensing circuits further comprise a fifth promoter operably linked to a nucleic acid encoding a second biomolecule that binds to and regulates the second promoter and is responsive to the first input signal.

In some embodiments, activity of the second biomolecule is induced by the first input signal.

In some embodiments, the copy of the first output molecule of (b) is fused to a protease recognition sequence.

In some embodiments, the protease recognition sequence is fused to a degradation tag.

In some embodiments, biosensing circuits further comprise a sixth promoter responsive to the second input signal and operably linked to a nucleic acid encoding a protease that cleaves the protease recognition sequence.

In some embodiments, the first input signal is peroxide. In some embodiments, the second input signal is paraquat. In some embodiments, the first promoter is a pLsoxS promoter. In some embodiments, the second promoter is an oxySp promoter. In some embodiments, the first biomolecule is SoxR. In some embodiments, the second biomolecule is OxyR.

In some embodiments, the protease is TevP.

Embodiments of the present disclosure provide cells comprising at least one biosensing circuit as provided herein.

In some embodiments, a cell endogenously expresses the first biomolecule, the second biomolecule, or both the first and the second biomolecule.

In some embodiments, the cell further comprises the first input signal, the second input signal, or both the first and second input signal.

Embodiments of the present disclosure provide methods of correcting crosstalk in a cell, comprising introducing into a cell at least one biosensing circuit as provided herein.

In some embodiments, a cell of the present disclosure is a bacterial cell (e.g., Escherichia coli cell).

These and other embodiments of the present disclosure are described in more detail herein.

The invention is not limited in its application to the details of construction and the arrangement of components set forth in the following description or illustrated in the drawings. The invention is capable of other embodiments and of being practiced or of being carried out in various ways. Each of the above embodiments may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. For purposes of clarity, not every component may be labeled in every drawing.

FIGS. 1A-1H show a hydrogen peroxide (H₂O₂)-sensing circuit. FIG. 1A shows an open-loop (OL) circuit used to screen H₂O₂-OxyR regulated promoters. OxyR is expressed from a constitutive pLlacO promoter on an medium copy plasmid (MCP), and mCherry is expressed from different promoters on a high copy plasmid (HCP). OxyR activation of mCherry expression is H₂O₂-dependent. OxyR is also expressed from the Escherichia coli (E. coli) genome and negatively regulates its own expression in an H₂O₂-independent manner. Dashed arrows are transcription-translation events and grey arrows are transcriptional regulation events. FIG. 1B illustrates an empirical H₂O₂-mCherry transfer function for three different promoters. The lines are Hill Equation fits to the raw data. The Hill Equations do not reach the theoretical maximum gene expression due to the toxicity of H₂O₂. FIG. 1C shows the sensitivity of the three different promoters calculated using the Hill Equation parameters from FIG. 1B. The maximum sensitivity of the oxySp promoter occurs at lower H₂O₂ concentrations than the other promoters. FIG. 1D represents the utility for the three different promoters calculated from the Hill Equations in FIG. 1B. The oxySp promoter had the highest utility. FIG. 1E shows an open-loop (OL) (top) and positive-feedback (PF) (bottom) H₂O₂-OxyR-oxySp circuits. In the open-loop circuit, oxyR is expressed from the proD promoter while the oxySp promoter controls mCherry expression; both occur on a high copy plasmid (HCP). In the positive-feedback circuit, an OxyR-mCherry fusion protein positively regulates its own expression from the oxySp promoter on a high copy plasmid. In both circuits, oxyR is also expressed from the E. coli genome and negatively regulates its own expression. FIG. 1F illustrates an empirical H₂O₂-mCherry transfer function for the open-loop and positive-feedback H₂O₂-OxyR-oxySp circuits. The lines are Hill Equation fits to the raw data. FIG. 1G shows the sensitivity of open-loop and positive-feedback H₂O₂-OxyR-oxySp circuits calculated using the Hill functions from FIG. 1F. FIG. 1H presents the utility for open-loop and positive-feedback H₂O₂-OxyR-oxySp circuits calculated using the Hill functions from FIG. 1F. The errors (s.e.m.) are derived from three flow cytometry experiments, each involving n=30,000 events.

FIGS. 2A-2H show a superoxide-sensing circuit. FIG. 2A shows positive-feedback (top) and open-loop (bottom) paraquat-SoxR-mCherry circuits. In the positive-feedback circuit, the pLsoxS promoter on an high copy plasmid controls the expression of a SoxR-mCherry fusion protein. In the open-loop circuit, soxR is constitutively expressed from pLlacO, a medium copy plasmid, and mCherry expression is controlled by the pLsoxS promoter on an high copy plasmid. SoxR is also expressed from the genome and negatively regulates its own expression. Dashed arrows are transcription-translation events and grey arrows are transcriptional regulation events. FIG. 2B illustrates an empirical paraquat-mCherry transfer function for paraquat-SoxR-mCherry positive-feedback and open-loop circuits. FIG. 2C demonstrates the sensitivity of the positive-feedback and open-loop circuits calculated using the Hill function from FIG. 2B. The sensitivity of the open-loop circuit is highest at every paraquat concentration. FIG. 2D presents the utility for the positive-feedback and open-loop circuits calculated from the Hill functions from FIG. 2B. The open-loop circuit has a higher utility. FIG. 2E shows an open-loop circuit in E. coli MG1655Pro. MG1655Pro constitutively expresses the lad repressor, which represses the pLlacO promoter and, thus, soxR expression from the MCP. Isopropyl β-D-1-thiogalactopyranoside (IPTG) dose-dependently inhibits Lad and derepresses pLlacO, inducing soxR expression from the medium copy plasmid. FIG. 2F demonstrates the empirical paraquat-mCherry transfer function for the paraquat-SoxR-mCherry open-loop circuits at different IPTG concentrations in MG1655pro. FIG. 2G represents the sensitivity functions derived from the Hill functions from FIG. 2F. FIG. 2H demonstrates the utility calculated from the Hill functions from FIG. 2F. The lowest concentration of IPTG, and thus SoxR, has the highest utility. The errors (s.e.m.) are derived from three flow cytometry experiments, each involving n=30,000 events.

FIGS. 3A-3H depict crosstalk correction in a dual-ROS sensing strain. FIG. 3A shows a first iteration of a dual-ROS sensing strain. SoxR is constitutively expressed from a low copy plasmid and activates mCherry expression from pLsoxS on a high copy plasmid. OxyR is constitutively expressed on a high copy plasmid and activates superfolder green fluorescent protein (sfGFP) expression from oxySp on the same high copy plasmid. Genomic soxR and oxyR are both autonegatively-regulated independent of their respective inducer concentrations. Dashed arrows are transcription-translation events and grey arrows are transcriptional regulation events. FIG. 3B illustrates the sfGFP output in terms of fold change relative to minimum fluorescence at different concentrations of H₂O₂ and paraquat. sfGFP expression is dependent upon H₂O₂ concentration, and there is little crosstalk with paraquat. FIG. 3C depicts the mCherry output in terms of fold change relative to minimum fluorescence for the dual-ROS sensing strain at different concentrations of H₂O₂ and paraquat. mCherry expression is mostly dependent upon paraquat concentration, but there is considerable crosstalk with H₂O₂ at high paraquat concentrations. FIG. 3D illustrates an analog correction component of the dual-ROS sensing strain. A copy of mCherry controlled by the oxySp promoter was added to the first iteration of the dual-ROS sensing strain. Consequently, it was determined that the expression of mCherry is dependent upon oxySp and pLsoxS promoter activity. FIG. 3E shows the mCherry output from a strain containing an analog correction component. The analog correction component over-corrects the H₂O₂ crosstalk at high paraquat concentrations, and significantly increases crosstalk at low paraquat concentrations. FIG. 3F demonstrates variable analog correction of the dual-ROS sensing strain. A medium copy plasmid mCherry gene that is under the transcriptional control of the oxySp promoter and is translationally fused to TEVrs and an LAA degradation tag was added to the first iteration of the dual-ROS-sensing strain. The tevP gene on a low copy plasmid under the control of the pLsoxS promoter was also added. TevP post-translationally cleaves the LAA degradation sequence from the oxySp-expressed mCherry protein at the TEVrs site, stabilizing the mCherry protein. Solid black arrows are post-translational events. FIG. 3G depicts the mCherry output from the Variable Analog Correction strain. H₂O₂ crosstalk is significantly reduced at high paraquat concentrations compared to the original dual-ROS sensing strain without increased crosstalk at low paraquat concentrations. FIG. 3H shows the total relative mCherry error for the three dual-ROS sensing strains. The analog correction and variable analog correction components both significantly reduce crosstalk. The errors (s.e.m.) are derived from three flow cytometry experiments, each involving n=30,000 events. * indicates P<0.05, ** indicates P<0.005.

FIGS. 4A-4F delineate differences between wild-type and ROS-impaired bone-marrow-derived dendritic cells (BMDCs). The graph shows mean and standard deviation of the log of green fluorescent protein (GFP) fluorescence (FIG. 4A) and the log of mCherry fluorescence (FIG. 4B) for wild-type BMDC and Cybb^{-/-} BMDCs cultured with dual ROS-sensor E. coli for the indicated time points and measured on fluorescence-activated cell sorting (FACS) gated for live BMDCs. The significance for each experiment was calculated with a Welch-corrected T-test. * indicates P<0.05, ** indicates P<0.005. *** indicates P<0.0005. **** indicates P<0.0001. ns indicates P>0.05. FIG. 4C depicts the difference between the mean of wild-type BMDC and of Cybb^{-/-} BMDC GFP fluorescence or mCherry fluorescence at the indicated time points from FIGS. 4A and 4B. The GFP sensor is more sensitive at every time point. FIG. 4D presents the microscopy of the dual-ROS strain cultured with BMDCs at the indicated time points. Green is GFP and blue is DAPI. Diphenyleneiodonium (DPI) is a Nox2-Inhibitor. DPI knocks down GFP expression. FIG. 4E shows E. coli with constitutive mCherry expression and H₂O₂-inducible GFP cultured with wild-type BMDCs for the indicated time points and gated on live BMDCs. The gate shown is for mCherry-positive BMDCs. FIG. 4F demonstrates that the log of GFP fluorescence plotted against the log of mCherry fluorescence from the gated cells in FIG. 4E is linearly correlated at each time point; however, there does not appear to be a trend in slope across the time points.

FIGS. 5A-5D are graphs showing theoretical calculations. FIG. 5A shows the best fit Hill function fitted to raw data. FIG. 5B shows the calculated input dynamic range. FIG. 5C shows the sensitivity curve. FIG. 5D shows the area under the sensitivity curve representing the 10% and 90% relative maxima.

FIGS. 6A and 6B show the utility metric simulated (FIG. 6A) over values of Bmax, C, and n (FIG. 6B).

FIGS. 7A-7C show the genomic soxR circuit. In FIG. 7A, SoxR is expressed from the genome and negatively regulates its own expression. mCherry expression is controlled by the pLsoxS promoter on a HCP. FIG. 7B depicts the empirical paraquat-mCherry transfer function for the genomic soxR circuit. FIG. 7C shows the sensitivity of the genomic soxR circuit.

FIGS. 8A-8D are maps of cross-talk errors depending on concentration of paraquat and H₂O₂. FIG. 8A shows the raw cross-talk error. The gene expression at a given paraquat concentration and zero H₂O₂ is overlaid on gene expression at the given paraquat and varying H₂O₂ concentrations. The difference between these two gene expression outputs is the raw cross-talk error. The graphs depict the raw cross-talk error (FIG. 8B), the absolute cross-talk error (FIG. 8C) and the relative cross-talk error (FIG. 8D).

FIG. 9 shows the absolute mCherry error for the first iteration of the dual-ROS sensing circuit (FIG. 3A) calculated from the mCherry output in terms of fold change relative to minimum fluorescence (FIG. 3C).

FIG. 10 depicts the relative mCherry error for the first iteration of the dual-ROS sensing circuit (FIG. 3A) calculated from the mCherry output in terms of fold change relative to minimum fluorescence (FIG. 3C).

FIG. 11 is a bar graph showing the total relative sfGFP error for the three dual-ROS sensing strains. There is not a significant difference between any of the circuits. The errors (s.e.m.) are derived from three flow cytometry experiments, each involving n=30,000 events. “ns” indicates P>0.05.

FIG. 12 shows the relative mCherry error for the analog correction dual-ROS sensing circuit (FIG. 3D) calculated from the mCherry output in terms of fold change relative to minimum fluorescence (FIG. 3E). Cross-talk is minimized at high concentrations of paraquat, but is much higher at low concentrations of paraquat compared to the initial dual-ROS sensing strain (FIG. 9).

FIG. 13A shows a biosensing circuit with a variable analog correction component without pLsoxS-tevP. mCherry expressed from oxySp is targeted for degradation due to the LAA degradation signal. FIG. 13B illustrates the mCherry output in terms of fold change relative to minimum fluorescence at different concentrations of H₂O₂ and paraquat. Because oxySp-mCherry is degraded, the mCherry output looks similar to the first iteration of the dual-ROS sensing strain (FIG. 3C).

FIG. 14A shows a biosensing circuit with a variable analog correction component without pLsoxS-mCherry. The mCherry output is only a function of mCherry expressed from the oxySp promoter. FIG. 14B shows the mCherry output in terms of fold change relative to minimum fluorescence at different concentrations of H₂O₂ and paraquat. This “corrective” function looks similar to the absolute mCherry error for the first iteration of the dual-ROS sensing strain (FIG. 9). FIG. 14C illustrates the mCherry output from FIG. 14B in two-dimensional terms; fold change of mCherry expression is plotted against the concentration of paraquat at different concentrations of H₂O₂. This plot reveals the potentiometric control of the H₂O₂-mCherry transfer function by paraquat.

FIG. 15 shows the relative mCherry error for the variable analog correction dual-ROS sensing circuit (FIG. 3F) calculated from the mCherry output in terms of fold change relative to minimum fluorescence (FIG. 3G). Cross-talk is much lower at high paraquat concentrations and is not considerably increased at low paraquat concentrations compared to the initial dual-ROS sensing strain (FIG. 10).

FIG. 16A represents the first iteration of the dual-ROS sensing strain. sfGFP output is dependent upon H₂O₂ input concentration and mCherry output is dependent upon paraquat input concentration. FIG. 16B shows the analog correction dual-ROS sensing strain. sfGFP output is dependent upon H₂O₂ input concentration. mCherry output is the sum of H₂O₂ input concentration and paraquat input concentration. FIG. 16C depicts a biosensing circuit having a variable analog correction component. sfGFP output is dependent upon H₂O₂ input concentration. mCherry output is dependent upon the sum of H₂O₂ input concentration and paraquat input concentration. The flux of mCherry from H₂O₂ is dampened by a potentiometer. The potentiometer takes paraquat concentration as an input to alter resistance.

FIG. 17A shows the circuit used to simultaneously track phagocytotic BMDCs and H₂O₂ concentration. FIG. 17B illustrates the relationship between mCherry and GFP fluorescence across all BMDCs.

FIG. 18 depicts the topology of gene regulatory networks as perceived from transcription factor-DNA interaction is contrasted with the behavior of these networks. The top row is an example of a network with crosstalk. The bottom row does not have crosstalk due to a crosstalk correction component.

### DETAILED DESCRIPTION OF THE INVENTION

A useful class of analog circuits includes those that exhibit high sensitivity across a wide input dynamic range, which are quantified with a metric called “utility.” Utility combines measures of the input dynamic range, output fold-induction, and sensitivity into a single value, which allows for the comparison of multiple gene circuit performances. Ideal biosensing circuits should also exhibit high specificity to desired analytes, but often suffer from crosstalk with non-cognate inputs. The present disclosure addresses the above challenges by providing a method to quantify and correct crosstalk between inputs via gene circuits that introduce tunable counter-crosstalk. As an illustrative example of one aspect of the present disclosure, the utility metric was used to guide the engineering of gene circuits in bacterial cells that sense reactive oxygen species (ROS), and a crosstalk correction method was used to deconvolute ROS input crosstalk. The ROS biosensing circuits can, for example, distinguish between wild-type dendritic cells and those with mutations in their ROS pathways.

Embodiments of the present disclosure provide biosensing circuits that include, among other components (e.g., genetic components), an analog correction component. A “biosensing circuit” refers to a circuit that detects and integrates multiple (e.g., 2, 3, 4 or more) environmental signals (e.g., chemical or non-chemical)—referred to as “input signals”—and generates a response (e.g., activates gene expression/production of a product) in a cell. Having multiple inputs in a cell can lead to crosstalk, by which a signal detected by one component in the circuit creates an undesired effect on another component in the circuit. Provided herein is an “analog correction component” that corrects crosstalk between two (or more) different signals in a cell by introducing counter crosstalk that “cancels out” any undesired effect.

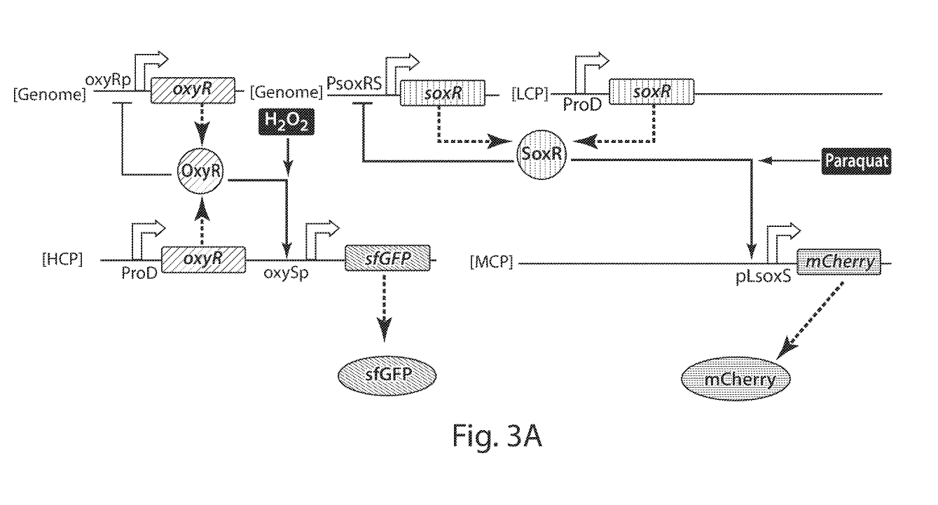

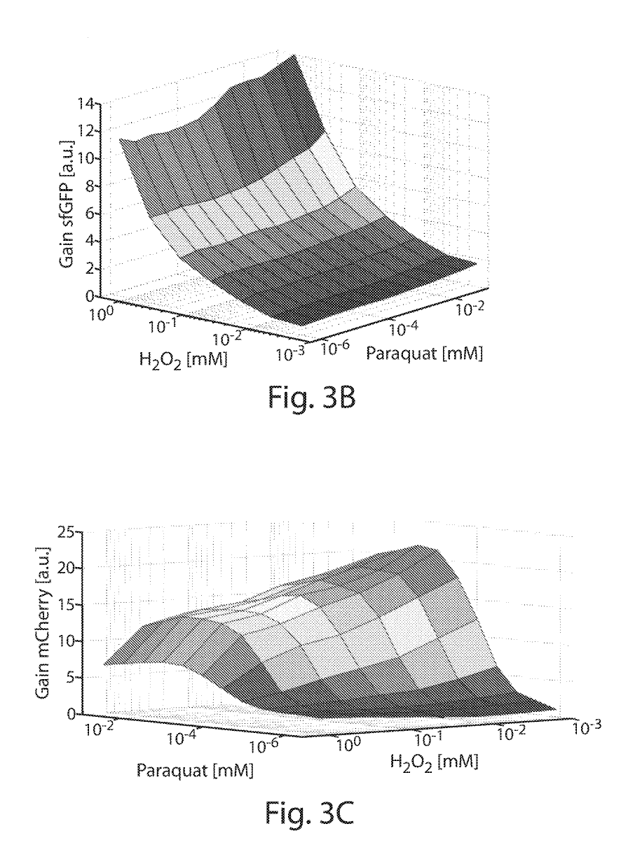

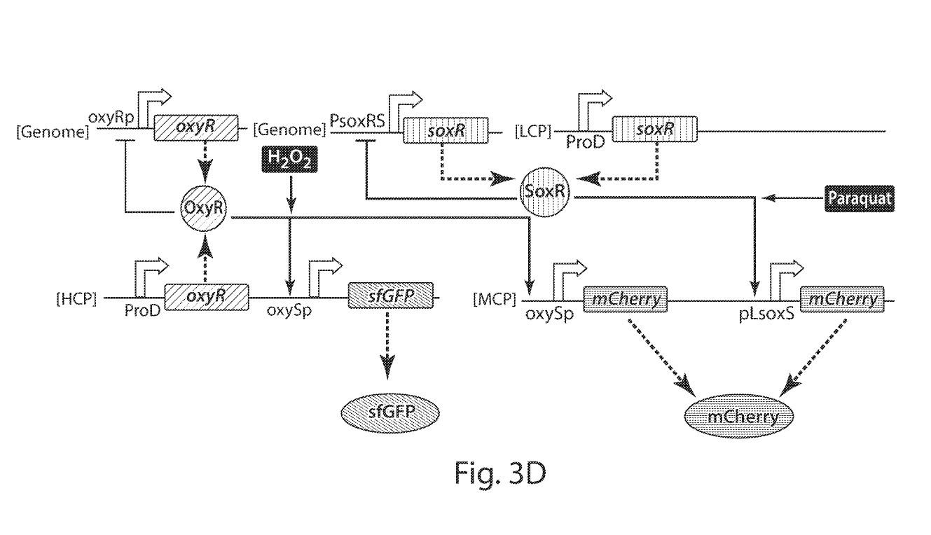

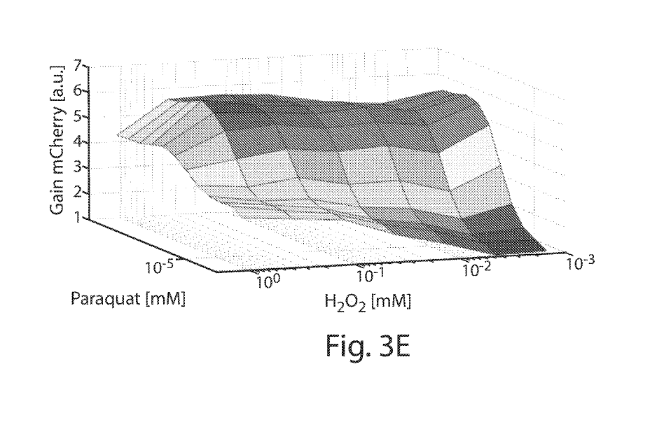

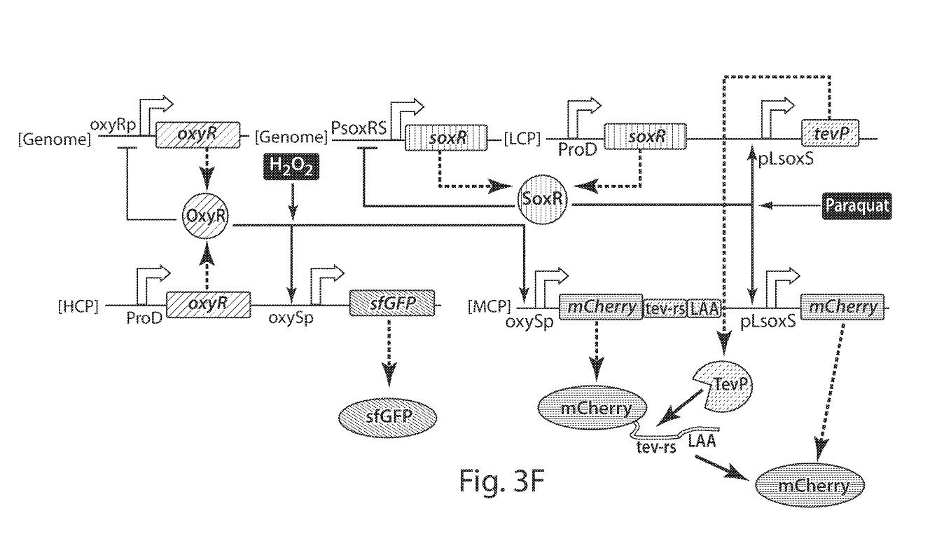

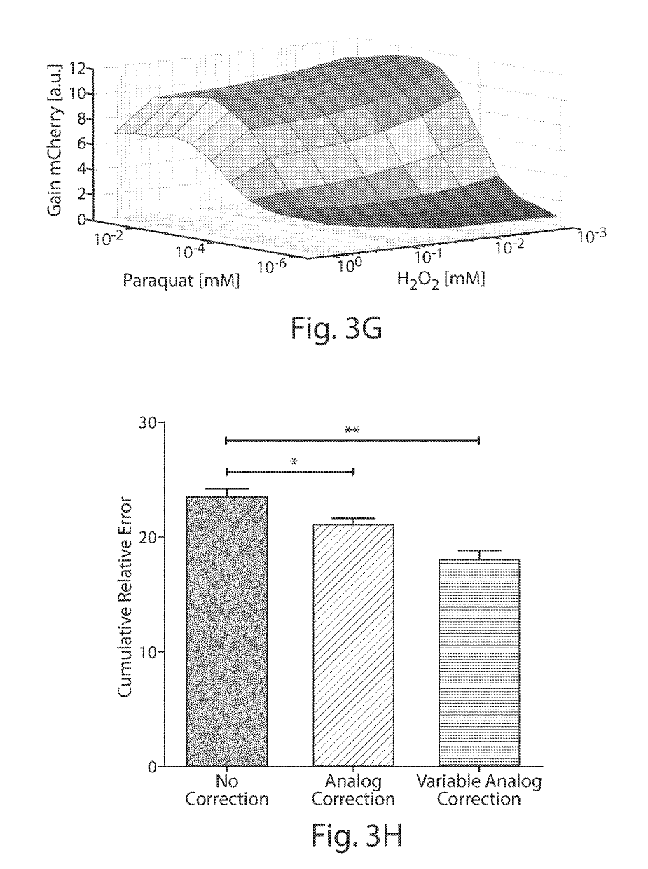

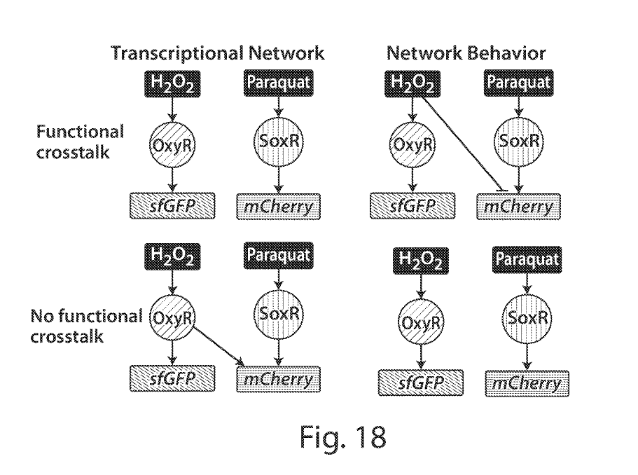

FIG. 3A depicts an example of a biosensing circuit for the detection of hydrogen peroxide (H₂O₂) and paraquat. The OxyR protein is a positive regulator of hydrogen peroxide-inducible genes in Escherichia coli and Salmonella typhimurium. Activity of the OxyR protein is modulated by hydrogen peroxide, and upon activation, binds to the oxySp promoter to initiate transcription of superfolded green fluorescent protein (sfGFP) (FIG. 18, top left). Likewise, activity of the SoxR protein (a redox-sensitive transcriptional regulator) is modulated by a superoxide radical-generating agent, paraquat, and upon activation, binds to the pLsoxS promoter to initiate transcription of mCherry (FIG. 18, top left). However, hydrogen peroxide has an undesired effect on the pLsoxS promoter, resulting is a decrease in expression mCherry (FIG. 18, top right). To negate this undesired effect, an analog correction component of the biosensing circuit can be introduced into the cell, as shown in FIG. 3D. For example, an oxySp promoter operably linked to a second copy of mCherry and having an opposite response to hydrogen peroxide, relative to the pLsoxS promoter, can be used to correct crosstalk by the hydrogen peroxide on the component that detects hydrogen peroxide and the component that detects paraquat. That is, the analog correction component (e.g., oxySp-mCherry), which produces mCherry in the presence of hydrogen peroxide, compensates for the undesired decrease in mCherry production that results from exposure of the pLsoxS promoter to hydrogen peroxide. Thus, the undesired effects of hydrogen peroxide on the pLsoxS promoter are effectively countered (or “canceled out”). The result is a response accurately reflective of the levels of hydrogen peroxide and paraquat in the cell. The relative level of mCherry produced in the cell is not affected by hydrogen peroxide crosstalk and, instead, is indicative only of the level of paraquat. While the above example is directed to hydrogen peroxide and paraquat input signals, the methods, circuits and components of the present disclosure are widely applicable for sensing and generating responses to myriad input signals for a variety of purposes.

Biosensing circuits of the present disclosure comprise promoters responsive to an (at least one) input signal and operably linked to a nucleic acid encoding an (at least one) output molecule. A “promoter” is a control region of a nucleic acid at which initiation and rate of transcription of the remainder of a nucleic acid are controlled. A promoter may also contain sub-regions at which regulatory proteins and other molecules, such as transcription factors, bind. Promoters of the present disclosure may be constitutive, inducible, activatable, repressible, tissue-specific, developmental stage-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid.

A promoter is considered “responsive” to an input signal if the input signal modulates (e.g., activates or inactivates, increases or decreases) the function of the promoter, indirectly or directly. In some embodiments, an input signal may positively modulate a promoter such that the promoter activates, or increases (e.g., by a certain percentage or degree), transcription of a nucleic acid to which it is operably linked. In some embodiments, by contrast, an input signal may negatively modulate a promoter such that the promoter is prevented from activating or inhibits, or decreases, transcription of a nucleic acid to which it is operably linked. In some embodiments, an input signal may inactivate a previously-active promoter. An input signal may modulate the function of the promoter directly by binding to the promoter or by acting on the promoter with or without an intermediate signal. For example, the OxyR protein (herein considered a “biomolecule”) modulates the oxySp promoter by binding to a region of the oxySp promoter. Thus, the OxyR protein is herein considered an input signal that directly modulates the oxySp promoter. By contrast, an input signal is considered to modulate the function of a promoter indirectly if the input signal modulates the promoter via an intermediate signal. For example, hydrogen peroxide modulates the OxyS protein, which, in turn, modulates the oxySp promoter. Thus, hydrogen peroxide is herein considered an input signal that indirectly modulates the oxySp promoter.

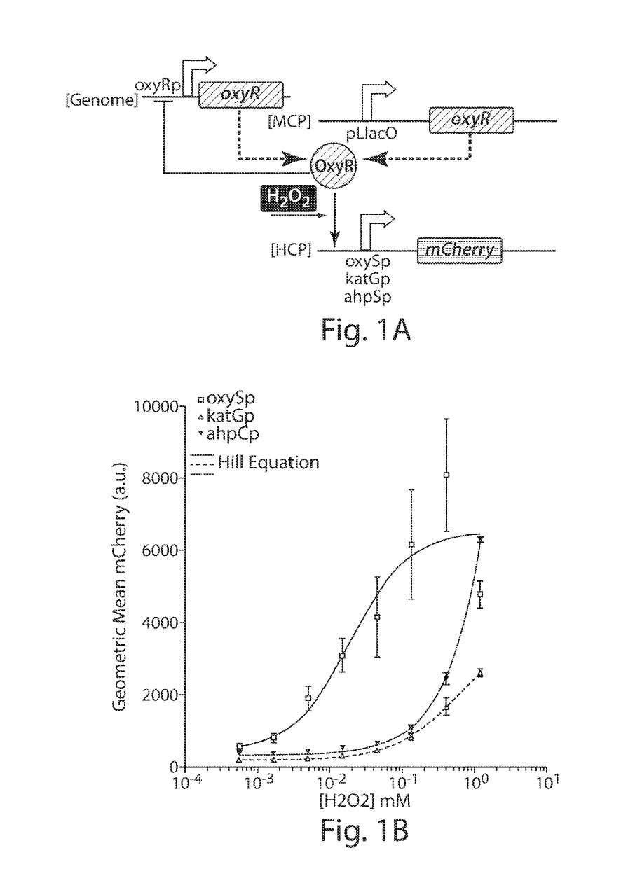

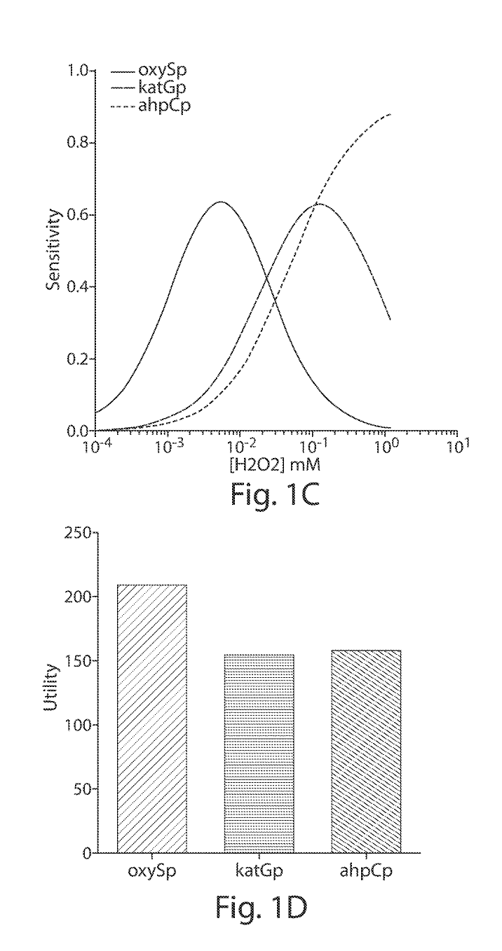

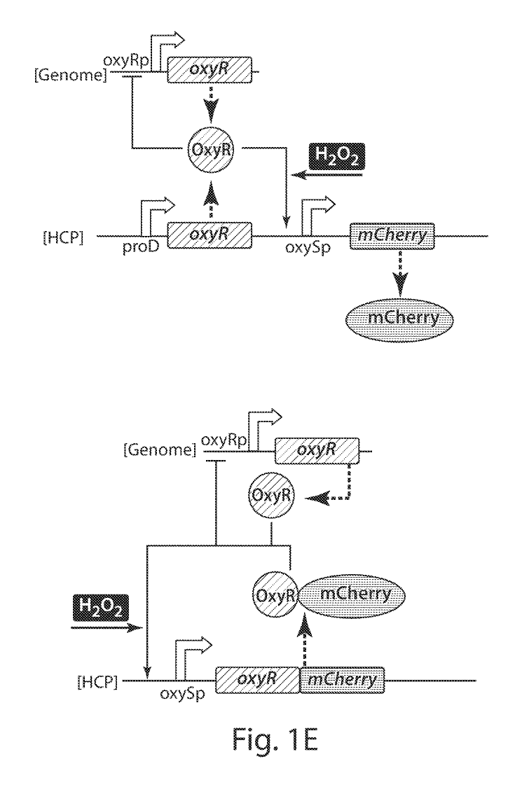

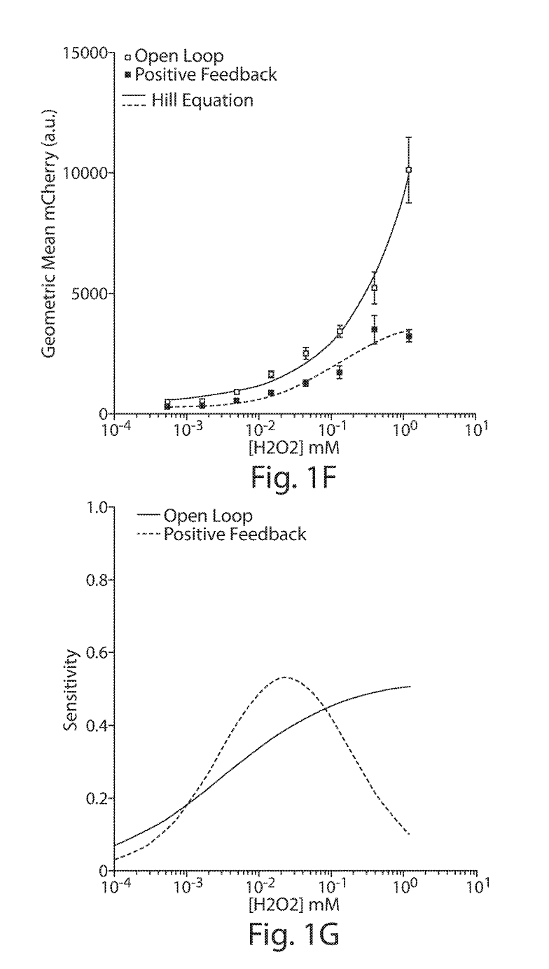

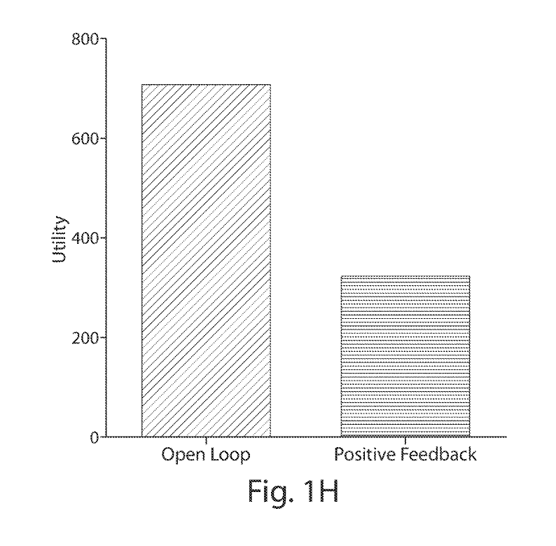

An “input signal” refers to any chemical signal (e.g., small molecule) or non-chemical signal (e.g., physical signal, such as light or heat) in a cell, or to which the cell is exposed, that modulates, directly or indirectly, a component (e.g., a promoter or enhancer) of a biosensing circuit. In some embodiments, an input signal is a biomolecule that directly modulates the function of a promoter by binding to the promoter or a nearby promoter element (referred to as direct modulation). In some embodiments, an input signal is a biomolecule that modulates another biomolecule, which then modulates (e.g., binds to and activates) the function of the promoter (referred to as indirect modulation). A “biomolecule” is any molecule that is produced in a live cell, e.g., endogenously or via recombinant-based expression. For example, with reference to FIG. 1E, hydrogen peroxide (H₂O₂) indirectly activates transcription of mCherry via its activation of OxyR and subsequent binding of OxyR to the oxySp promoter. Thus, hydrogen peroxide is a biomolecule input signal that indirectly modulates the oxySp promoter and, in turn, expression of mCherry. Likewise, the OxyR protein is itself considered a biomolecule input signal because it directly modulates transcription of mCherry by binding to the oxySp promoter. In some embodiments, an input signal may be endogenous to a cell or a normally exogenous condition, compound or protein that contacts a promoter of a biosensing circuit in such a way as to be active in modulating (e.g., inducing or repressing) transcriptional activity from a promoter responsive to the input signal (e.g., an inducible promoter). It should be understood that input signals are not limited to biomolecules, as discussed above. It should also be understood that hydrogen peroxide and OxyR are examples of biomolecules that may be used in accordance with the present disclosure. Other biomolecules may be used. Likewise, input signals are not limited to biomolecules. Synthetic molecules and chemical molecules (e.g., small molecule chemicals/drugs), for example, may also be used, as discussed below.

Examples of chemical input signals include, without limitation, signals extrinsic or intrinsic to a cell, such as amino acids and amino acid analogs, saccharides and polysaccharides, nucleic acids, protein transcriptional activators and repressors, cytokines, toxins, petroleum-based compounds, metal containing compounds, salts, ions, enzymes, enzyme substrates, enzyme substrate analogs, hormones and quorum-sensing molecules.

Examples of non-chemical input signals include, without limitation, changes in physiological conditions, such as changes in pH, light, temperature, radiation, osmotic pressure and saline gradients.

Promoters of the present disclosure that are responsive to an input signal may be considered “inducible” promoters. Inducible promoters for use in accordance with the present disclosure include any inducible promoter described herein or known to one of ordinary skill in the art. Examples of inducible promoters include, without limitation, chemically-regulated, biochemically-regulated and physically-regulated promoters, such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Biosensing circuits, in some embodiments, are designed to detect and generate a response to one or multiple input signals. For example, a biosensing circuit may detect and generate a response to 2, 3, 4, 5, 6, 7, 8, 9 or 10 input signals. Similarly, the present disclosure provides biosensing circuits having multiple output molecules (e.g., 2 to 10 output molecules).

Biosensing circuits of the present disclosure, in some embodiments, generate a response in the form of an output molecule. An “output molecule” refers to any detectable molecule (e.g., detectable molecule) under the control of (e.g., produced in response to) an input signal. For example, as shown in FIG. 3A, sfGFP is an output molecule produced in response to activation of OxyR by hydrogen peroxide. Likewise, mCherry is an output molecule produced in response to activation of SoxR by paraquat.

Examples of output molecules include, without limitation, proteins and nucleic acids.

Examples of output protein molecules include, without limitation, marker proteins such as fluorescent proteins (e.g., GFP, EGFP, sfGFP, TagGFP, Turbo GFP, AcGFP, ZsGFP, Emerald, Azami green, mWasabi, T-Sapphire, EBFP, EBFP2, Azurite, mTagBFP, ECFP, mECFP, Cerulean, mTurquoise, CyPet, AmCyan1, Midori-ishi Cyan, TagCFP, mTFP1, EYFP, Topaz, Venus, mCitrine, YPET, TagYFP, PhiYFP, ZsYellow1, mBanana, Kusabira Orange, Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143 and variants thereof), enzymes (e.g., catalytic enzymes such as recombinases, caspases), biosynthetic enzymes, cytokines, antibodies, regulatory proteins such as transcription factors, polymerases and chromatin remodeling factors.

Examples of output nucleic acid molecules include, without limitation, RNA interference molecules (e.g., siRNA, miRNA, shRNA), guide RNA (e.g., single-stranded guide RNA), trans-activating RNAs, riboswitches, ribozymes and RNA splicing factors.

Biosensing circuits may contain one or multiple (e.g., 2, 3, 4 or more) copies of an output molecule. In some embodiments, each copy is operably linked to a different promoter. For example, FIG. 3D shows an example of an analog correction component that contains two copies of mCherry, one operably linked to the oxySp promoter, and the other operably linked to the pLsoxS promoter. An “analog correction component” of a biosensing circuit refers to two different promoters in the circuit, each operably linked to a copy of the same gene, which respond in opposite ways to the same input. For example, if a first promoter (e.g., pLsoxS) operably linked to a first copy of a gene (e.g., mCherry), in response to a input signal (e.g., H₂O₂), inhibits expression of the first copy of the gene, and a second promoter (e.g., oxySp) operably linked to a first copy of a gene (e.g., mCherry), in response to the same signal (e.g., H₂O₂), activates expression of the second copy of the gene, then collectively, the two promoters are considered “an analog correction component” of the biosensing circuit. In the example shown in FIG. 3D, the oxySp promoter operably linked to one copy of mCherry and the pLsoxS operably linked to another copy of mCherry are collectively considered “an analog correction component” of the depicted biosensing circuit. The oxySp promoter and the pLsoxS promoter, each operably linked to a copy of the same gene, respond in opposite ways to H₂O₂.

It should be understood that different components of a biosensing circuit may produce one or more copy(ies) of an output molecule. Reference to an output molecule produced in a cell as a response generated by a biosensing circuit accounts for the collective (sum) production of all copies of the output molecule in the cell, unless indicated otherwise. For example, some biosensing circuits may contain (a) a first promoter (e.g., pLsoxS) responsive to a first (e.g., H₂O₂) and second (e.g., paraquat) input signal and operably linked to a nucleic acid encoding an output molecule (e.g., mCherry), and (b) second promoter (e.g., oxySp) responsive only to the first signal (e.g., H₂O₂) and operably linked to a to a nucleic acid encoding a copy of the output molecule (e.g., mCherry). In such circuits, the response of the second promoter (e.g., oxySp) to the first input signal (e.g., H₂O₂) is opposite the response of the first promoter (e.g., pLsoxS) to the first input signal (e.g., H₂O₂) such that the first input signal (e.g., H₂O₂) does not affect relative production of the “output molecule.” The “output molecule” refers to the total amount of output molecule produced in the cell—the sum of the output molecule of (a) and the copy of the output molecule of (b) (e.g., the sum of mCherry from (a) and sum of mCherry from (b); FIG. 3D).

Biosensing circuits of the present disclosure, in some embodiments, contain two (e.g., at least two) different promoters, each operably linked to a copy of the same output molecule and each responsive to the same input signal. In some embodiments, the responses of two promoters to the same input signal are “opposite to each other” such that the input signal does not affect relative production of the output molecule. For example, if one promoter activates transcription of the nucleic acid to which it is operably linked, then the other promoter, having an opposite response, deactivates transcription of the nucleic acid to which it is operably linked. In this manner, the relative response—the relative production of the output molecule—is independent of the input signal and, in some instances, may be modulated by another, second, input signal. With reference to FIG. 3D as an illustrative example, although the oxySp promoter and the pLsoxS promoter are both responsive to OxyR/hydrogen peroxide (H₂O₂), each responds oppositely relative to the other—oxySp responds positively to hydrogen peroxide to activate mCherry production, and pLsoxS responds negatively to hydrogen peroxide to inhibit mCherry production. pLsoxS also responds positively to paraquat to activate mCherry production. Thus, the level of mCherry compromised (or not produced) as a result of the negative modulation by hydrogen peroxide on pLsoxS is compensated for by the level of mCherry produced as a result of the positive modulation by hydrogen peroxide on oxySp. In this way, crosstalk by hydrogen peroxide is countered (also referred to as “corrected”). The relative production of mCherry, having crosstalk corrected, is now dependent primarily on paraquat.

In some embodiments, biosensing circuits contain two or more (e.g., 2, 3, 4 or more) differ output molecules (e.g., 2 or more different fluorescent proteins such as GFP and mCherry, or two or more different types of output molecules such as a transcription factor or small RNAs that control transcription and a fluorescent protein). In some embodiments, an output molecule regulates expression of another output molecule (e.g., is a transcription factor that regulates a promoter, which drives expression of another output molecule). For example, a first promoter may be operably linked to a first output molecule (e.g., transcription factor 1), and a second promoter may be linked to a second output molecule (e.g., transcription factor 2), wherein the first and second output molecules have opposite effects on the expression of a third output molecule (e.g., a fluorescent reporter molecule). That is, the first output molecule may upregulate expression of the third output molecule, while the second output molecule may downregulate expression of the third output molecule.

An input signal “does not affect” relative production of an output molecule if the relative level of the output molecule remains the same, or increases or decreases by less than 25% (or less that 20%, less than 15%, less than 10%, less than 5%), relative to the production level of the output molecule in the absence of the same input signal.

In some embodiments, to achieve a counter crosstalk effect in a biosensing circuit (e.g., in a live cell), an analog correction component can be “tuned” such that opposite responses to the same input signal are proportional (or at least substantially proportional, e.g., within 5%-10%, or within 5%, 6%, 7%, 8%, 9% or 10%) to each other. Tuning of a biosensing circuit may also be achieved, for example, by controlling the level of nucleic acid expression of particular components of the circuit. This control can be achieved, for example, by controlling copy number of the nucleic acids (e.g., using low, medium and/or high copy plasmids, and/or constitutively-active promoters) (see, e.g., FIG. 3D), adjusting the translation rate or transcription rate and/or adjusting the degradation rate.

Biosensing circuits may also be tuned by modulating the stability of an output protein. For example, as shown in FIG. 3F, a protease recognition sequence (e.g., tev-rs) and degradation tag (LAA) may be fused to a copy of the output protein (e.g., mCherry). A nucleic acid encoding the cognate protease (e.g., TevP) is operably linked to a promoter (e.g., pLsoxS) responsive to an input signal (e.g., paraquat). In this manner, stability of the output molecule is dependent on the concentration of the input signal.

Promoters that respond opposite to each other may be on the same vector (e.g., plasmid) or on different vectors (e.g., each on a separate plasmid). In some embodiments, promoters that respond opposite to each other may be on the same vector high copy plasmid, medium copy plasmid, or low copy plasmid.

For clarity and ease of explanation, promoters responsive to a signal may be referred to as first, second or third promoters (and so on) so as to distinguish one promoter from another. It should be understood that reference to a first promoter and a second promoter, unless otherwise indicated, is intended to encompass two different promoters (e.g., oxySp v. pLsoxS). Similarly, output molecules may be referred to as a first, second or third output molecules (and so on) so as to distinguish one output molecule from another. It should be understood that reference to a first output molecule and a second output molecule, unless otherwise indicated, is encompasses two different output molecules (e.g., GFP v. mCherry).

In some embodiments, production of an output molecule by a single component of a biosensing circuit may be increased as a result of a promoter of the component responding to an input signal. Production of an output molecule (or a copy of an output molecule) of a component is considered to be “increased” if the level of the output molecule (or a copy of an output molecule) produced in response to a input signal is greater than the level of the output molecule produced in the absence of the same input signal. In some embodiments, production of an output molecule (or a copy of an output molecule) is considered to be increased if the level of the output molecule (or a copy of an output molecule) produced in response to a input signal is at least 5%, at least 10%, at least 15%, at least 20%, or at least 25% greater than the level of the output molecule (or a copy of an output molecule) produced in the absence of the same input signal.

Biosensing circuits of the present disclosure may be used to detect more than one input signal in a cell. In such embodiments, a biosensing circuit may comprise, in addition to an analog correction component, a component that detects and generates a response to a first input signal and a component that detects and generates a response to a second input signal. The component that detects the first input signal may contain a promoter responsive to the first input signal and operably linked to a first output molecule (e.g., GFP). The component that detects the second input signal may contain a promoter responsive to the second input signal and operably linked to a second output molecule (e.g., mCherry) that is different from the first output molecule. In this way, an independent response to each signal may be generated.

Thus, in some embodiments, a biosensing circuit comprises (a) a first promoter responsive to a first input signal and operably linked to a nucleic acid encoding a first output molecule, (b) a second promoter responsive to the first input signal and operably linked to a nucleic acid encoding a copy of the first output molecule, and (c) a third promoter responsive to the first input signal and operably linked to a nucleic acid encoding a second output molecule that is different from the first output molecule, wherein the response of the second promoter to the first input signal is opposite the response of the first promoter to the first input signal such that the first input signal does not affect relative production of the first output molecule.

Biosensing circuits of the present disclosure may be expressed in a broad range of host cell types. Biosensing circuits may be expressed, for example, in a prokaryotic cell or a eukaryotic cell. In some embodiments, biosensing circuits are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, biosensing circuits are expressed in mammalian cells. For example, in some embodiments, biosensing circuits are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSYSY human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure are generally considered to be modified. A modified cell is a cell that contains an exogenous nucleic acid or a nucleic acid that does not occur in nature (e.g., a biosensing circuit of the present disclosure). In some embodiments, a modified cell contains a mutation in a genomic nucleic acid. In some embodiments, a modified cell contains an exogenous independently replicating nucleic acid (e.g., components of biosensing circuits present on an episomal vector). In some embodiments, a modified cell is produced by introducing a foreign or exogenous nucleic acid into a cell. Thus, provided herein are methods of introducing a biosensing circuit into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination).

In some embodiments, a cell contains a gene deletion.

Biosensing circuits of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., a biosensing circuit or component thereof) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Provided herein are methods of correcting crosstalk in a cell that contains at least one (one, two, three or more) biosensing circuit. In some embodiments, biosensing circuits as provided herein may be used as a diagnostic tool to detect (“sense”) changes (e.g., biological, physiological or chemical changes) associated with a condition or disease stage. Thus, in some embodiments, provided herein are methods of delivering biosensing circuits (e.g., containing an analog correction component) to a subject (e.g., a human subject). Biosensing circuits may be delivered to subjects using, for example, in bacteriophage or phagemid vehicles, or other delivery vehicle that is capable of delivering nucleic acids to a cell in vivo. In some embodiments, biosensing circuits may be introduced into cells ex vivo, which cells are then delivered to a subject via injection, oral delivery, or other delivery route or vehicle.

Other uses of biosensing circuits are contemplated by the present disclosure. For example, the present disclosure provides cells engineered to dynamically control the synthesis of molecules or peptides based on intrinsic factors (e.g., the concentration of metabolic intermediates) or extrinsic factors (e.g., inducers); biosensing circuits engineered to classify a cell type (e.g., via inputs from outside of the cell, such as receptors, or inputs from inside of the cell, such as transcription factors, DNA sequence and RNAs); and cells engineered to synthesize materials in a spatial pattern based on, for example, environmental cues.

It should be understood that while biosensing circuits of the present disclosure, in many embodiments, are delivered to cells or are otherwise used in vivo, the invention is not so limited. Biosensing circuits as provided herein may be used in vivo or in vitro, intracellularly or extracellularly (e.g., using cell-free extracts/lysates). For example, biosensing circuits may be used in an in vitro abiotic paper-based platform as described in Pardee K et al. (Cell, Corrected Proof published online Oct. 23, 2014, in press, incorporated by reference herein) to, for example, enable rapid prototyping for cell-based research and gene circuit design.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

### Example 1

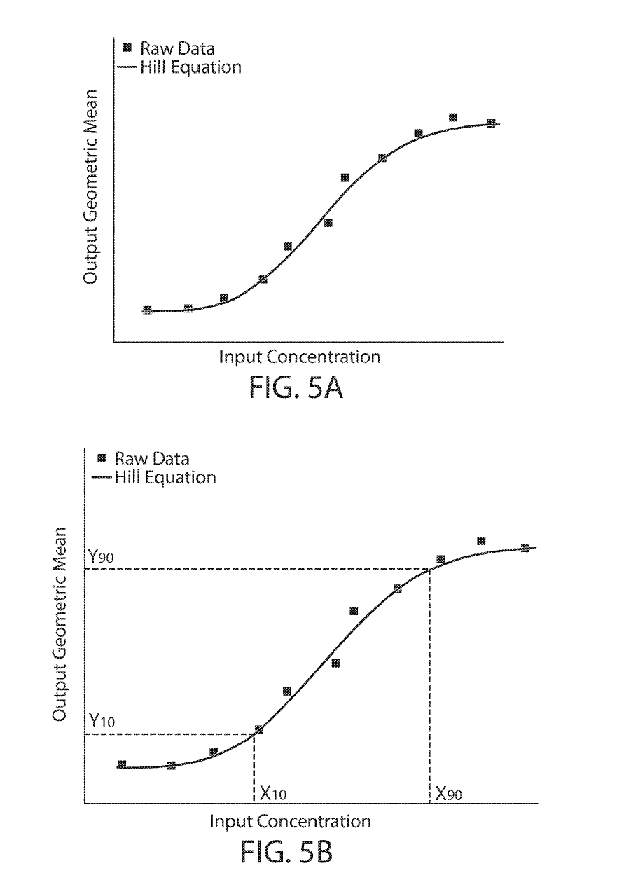

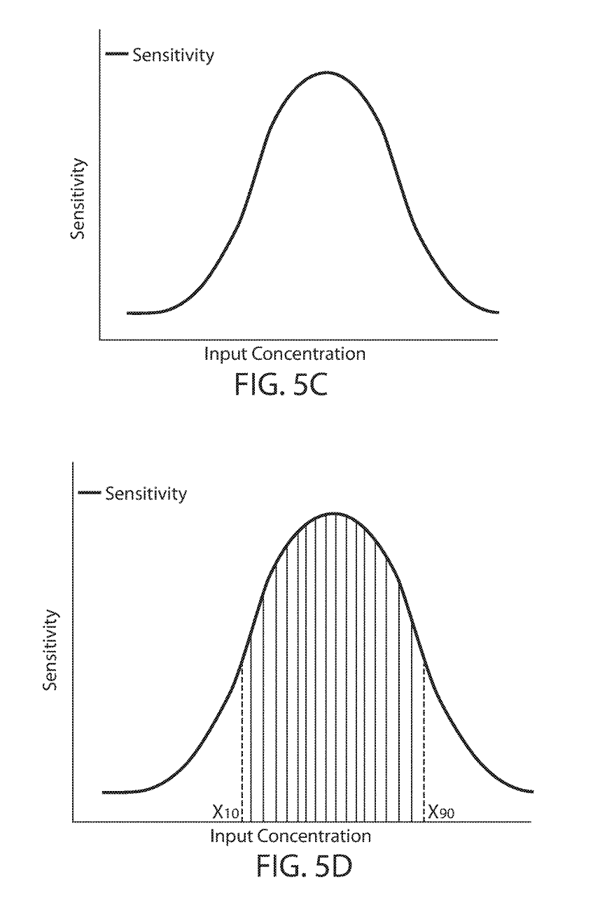

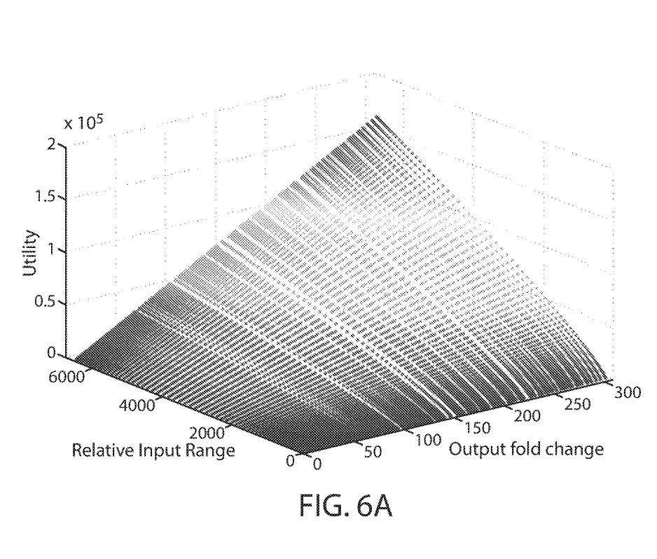

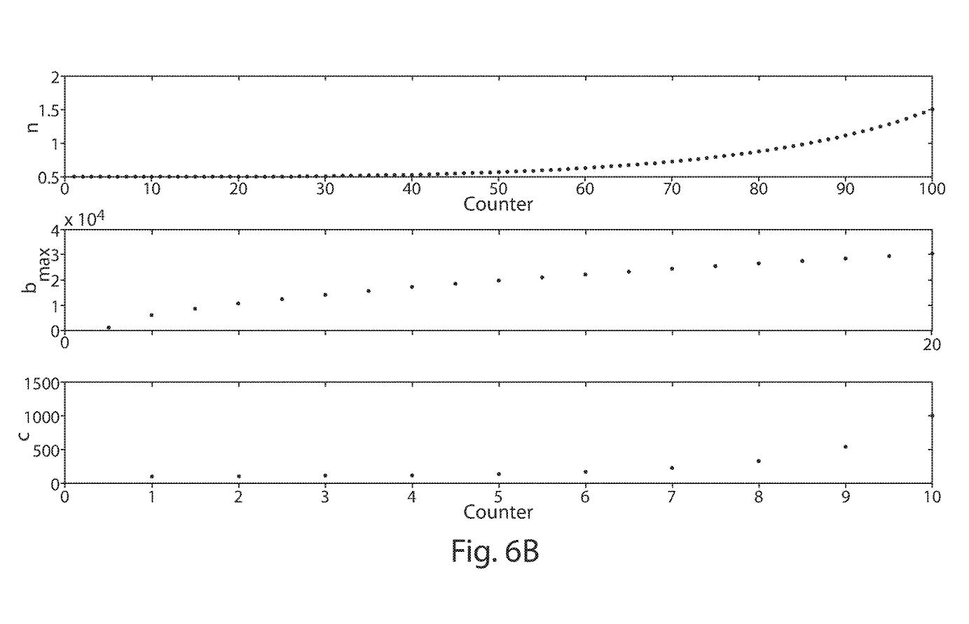

Gene circuits that measure the concentration of H₂O₂ based on the OxyR transcriptional activator in BW25113 Escherichia coli (E. coli) were first created. To determine the optimal H₂O₂-OxyR responsive promoter, open-loop (OL) gene circuits were built with oxyR constitutively expressed from a medium copy plasmid (MCP) and mCherry expression controlled by OxyR-activated promoters on a high copy plasmid (HCP) (FIG. 1A). Constitutive oxyR expression was used to decouple the circuit from endogenous feedback regulation. The raw empirical gene expression data were fitted to Hill functions (FIG. 1B), which were used to calculate the sensitivity of each circuit (FIG. 1C). The utility of each circuit was calculated (FIG. 1D) by integrating the sensitivity function over the input dynamic range, normalizing this integrand to the relative size of the input dynamic range, and multiplying by the output fold-induction (FIG. 5). Utility scales well over simulations with varying Hill equation parameters (FIGS. 6A and 6B). Extracellular H₂O₂ concentrations above 1.08 mM were toxic and therefore these concentrations were avoided in the experimental setup. In the range of H₂O₂ concentrations tested, the input-output functions did not saturate, so the observed maximum gene expression was used to calculate the output fold-induction and input dynamic range. The promoter from the small RNA oxyS (OxySp) outperformed the other oxyR-regulated promoters that had been previously utilized as biosensors. OxySp had both the second highest output fold-induction (15.0×) and the highest relative input range (58.4×). The utility was calculated to be 210.8, which was slightly higher than the utility of the katGp circuit (155.3) and ahpCp circuit (159.2) (FIG. 1D). Notably, the oxySp circuit's sensitivity was higher across lower H₂O₂ concentrations than the other promoters that were screened (FIG. 1C). This sensitivity range is useful for sensing physiologic concentrations of H₂O₂, which are reported to be 0.5-50 μM near wounds and 17 μM in neutrophil phagosomes in the absence of myeloperoxidase. The values are reportedly lower with myeloperoxidase.

Tuning OxyR production optimized the performance of the H₂O₂-OxyR-oxySp circuit. OxyR expression was increased in the open-loop (OL) circuit by constitutive production from a strong proD promoter on a high copy plasmid (HCP), in addition to the genomic oxyR (FIG. 1E). This increased the output-fold induction to 23.6× and the relative input range to 63.0×, resulting in a utility of 711.0. (FIG. 1H). In addition, oxyR was tested in a positive-feedback (PF) circuit by fusing mCherry to the carboxy terminus of oxyR and placing the composite gene under the control of oxySp (FIG. 1E). This circuit had a wider relative input range (72.5×) than the OL circuit but a significantly lower output fold change (15.9×), possibly due to a lower maximum concentration of oxyR in the cell. The utility for the PF circuit was 326.2.

### Example 2

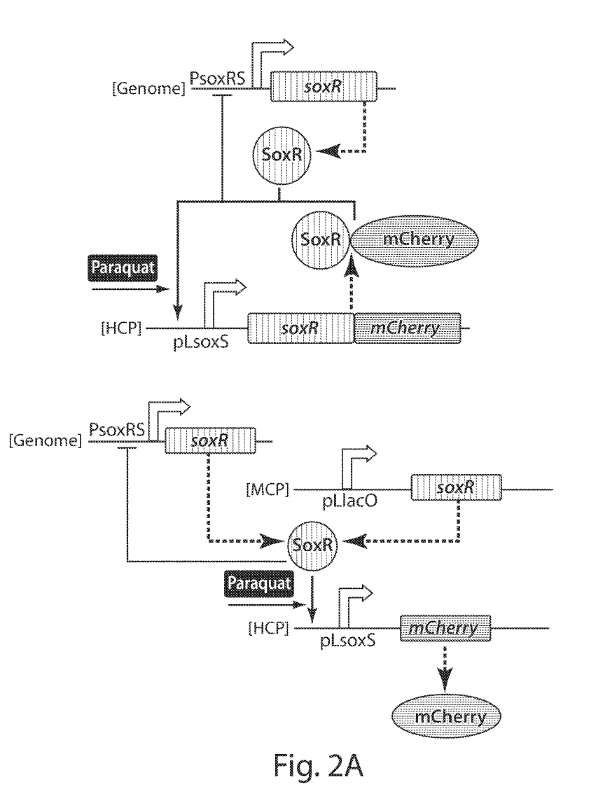

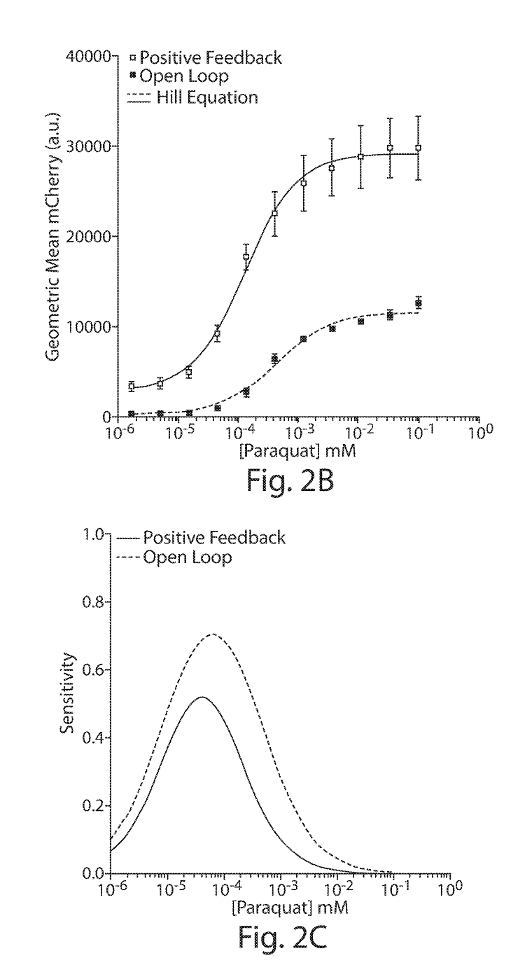

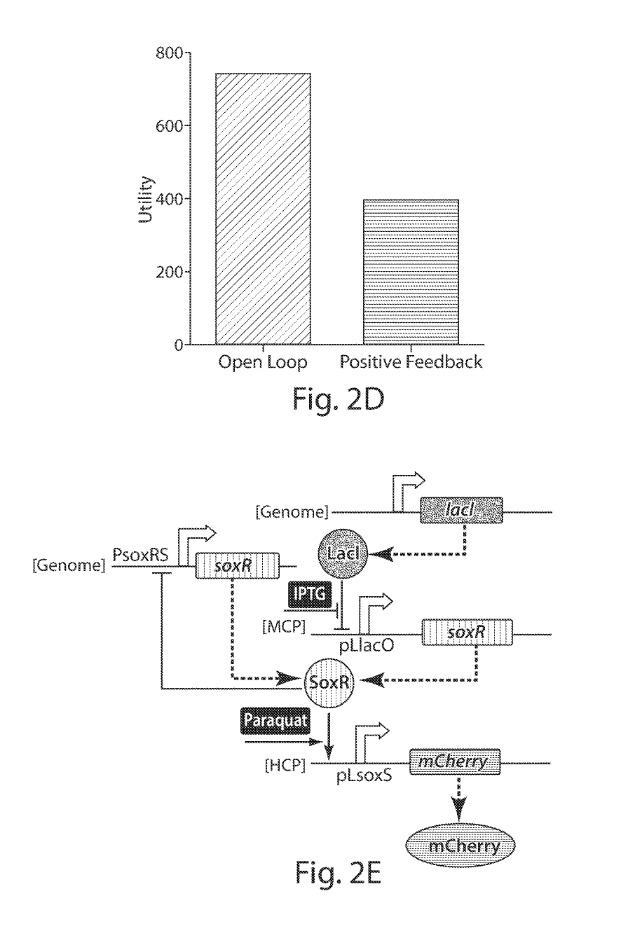

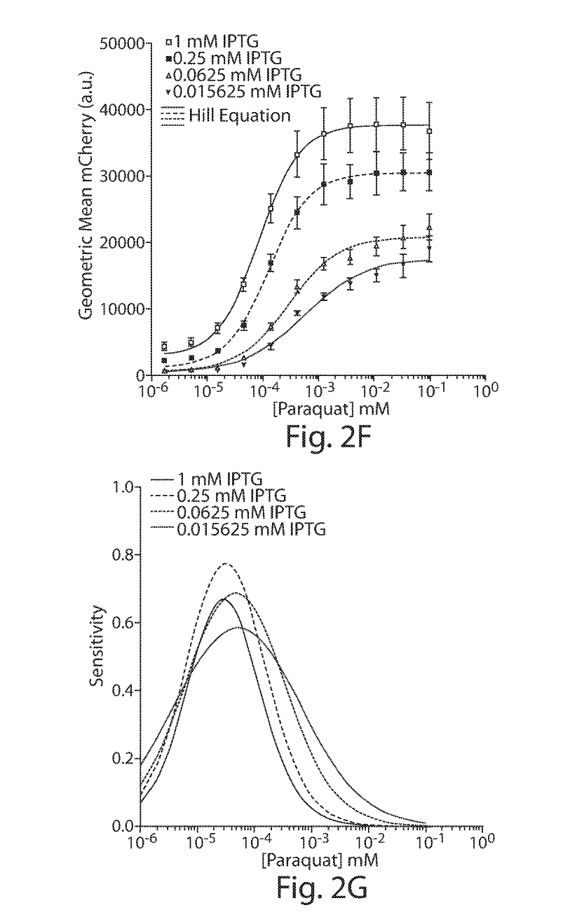

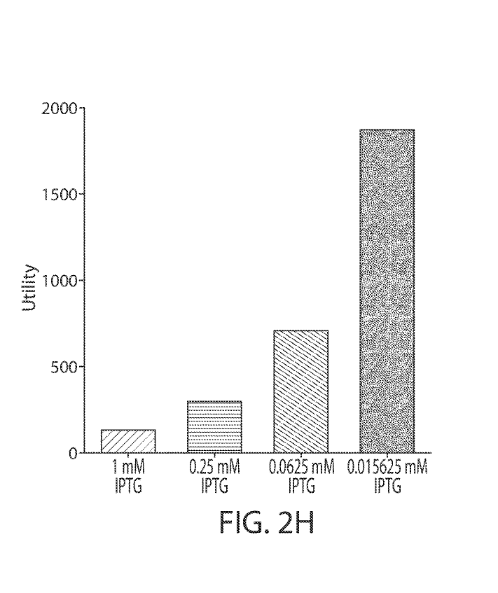

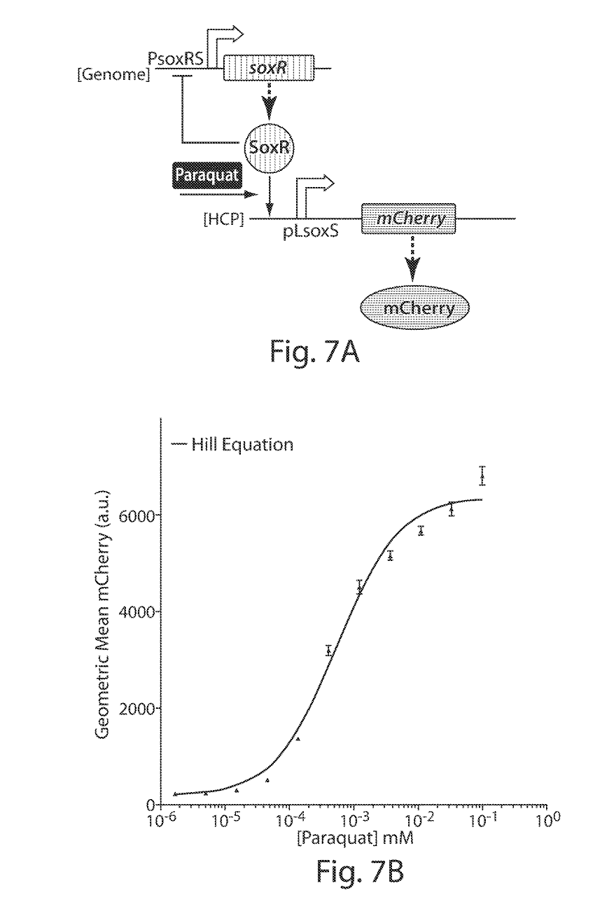

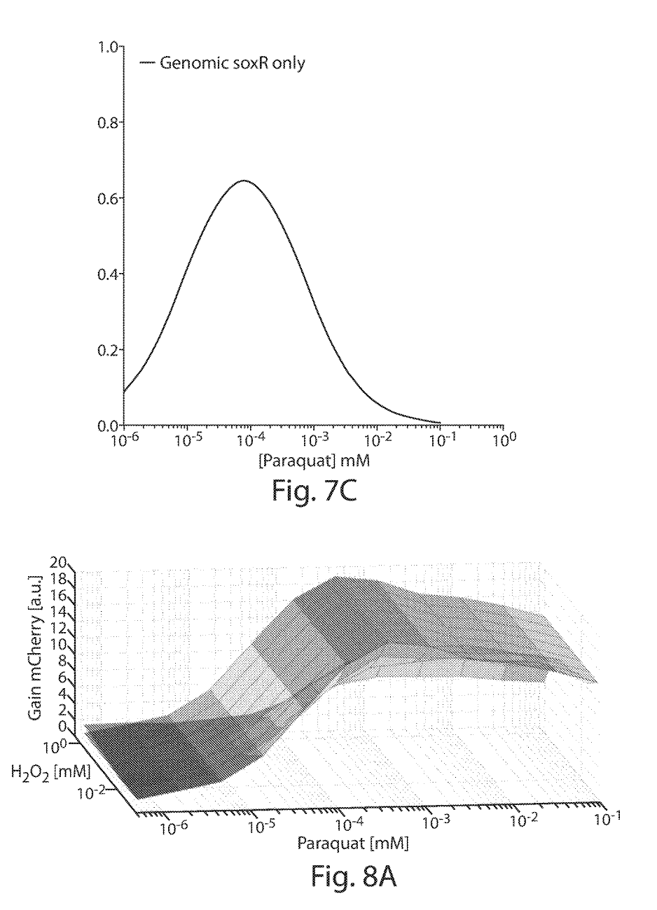

A superoxide-sensing circuit was created based on the SoxR transcriptional activator, which is reported to respond to superoxide and redox cycling-reagents such as paraquat. To express the output, the pLsoxS promoter was used. The promoter has a SoxR binding site from the soxS promoter fused to the lambda phage −35 and −10 promoter region. A positive-feedback (PF) circuit was built, with a soxR-mCherry fusion protein controlled by the pLsoxS promoter on a high copy plasmid (HCP), and an open-loop (OL) circuit was created, with soxR constitutively expressed from an medium copy plasmid (MCP) and mCherry expression controlled by the pLsoxS promoter on an HCP (FIG. 2A). The OL circuit had both a significantly larger output fold induction (42.3× vs. 10.2×) and relative input range (95.8× vs. 82.6×) than the PF circuit (FIG. 2B), resulting in a higher utility (891.9 vs. 169.1) (FIG. 2D). Given that SoxR binds to transcription factor-binding sites in its uninduced, reduced state (when paraquat is absent), it was postulated that decreasing the concentration of SoxR in the cell would improve circuit functionality since transcription factors that bind DNA target sites in a ligand-independent manner are often found at low copy numbers per genome. Indeed, a circuit where soxR was only expressed from its native promoter in the genome had an increased relative input range (126.02×), yet a decreased output fold induction (34.64×) and utility (870.74) (FIG. 7).

A low level of constitutive soxR expression optimized circuit performance. The OL circuit was transformed into an MG1655Pro E. coli strain that constitutively expresses the Lad repressor protein from the genome, which enabled control of soxR expression with the small molecule IPTG (FIG. 2E). The lowest IPTG concentration maximized circuit utility to 1881.9 (FIG. 2H). Lower concentrations of SoxR maximized both the output fold induction and relative input range, which are normally a trade-off in analog circuits.

### Example 3

A dual-ROS sensing E. coli strain that can measure the concentration of both paraquat and H₂O₂ was built based on the single-ROS sensor circuits. The paraquat-sensing circuit was in an open-loop (OL) configuration with soxR constitutively expressed from a low copy plasmid (LCP) and mCherry expression controlled by pLsoxS on a medium copy plasmid (MCP) (FIG. 3A). The H₂O₂-sensing circuit was in an OL conformation and fully encoded on a high copy plasmid (HCP) with sfGFP as an output. The dual-ROS sensor strain was exposed to 84 different combinations of concentrations of H₂O₂ and paraquat up to a maximum extracellular H₂O₂ concentration of 1.08 mM and paraquat concentration of 0.1 mM, and fluorescent reporter expression was measured via flow cytometry. Little crosstalk was found between paraquat and the H₂O₂-sensing circuit as sfGFP expression at any given H₂O₂ concentration was not considerably affected by paraquat (FIG. 3B). In contrast, the paraquat-sensing circuit was drastically (appreciably) affected by H₂O₂ as mCherry expression at high paraquat concentrations was dampened (reduced) by H₂O₂ (FIG. 3C).

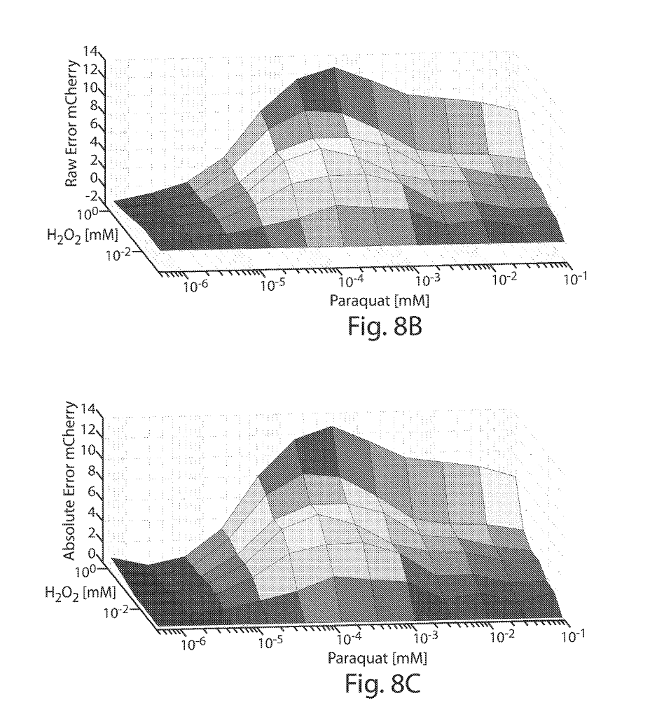

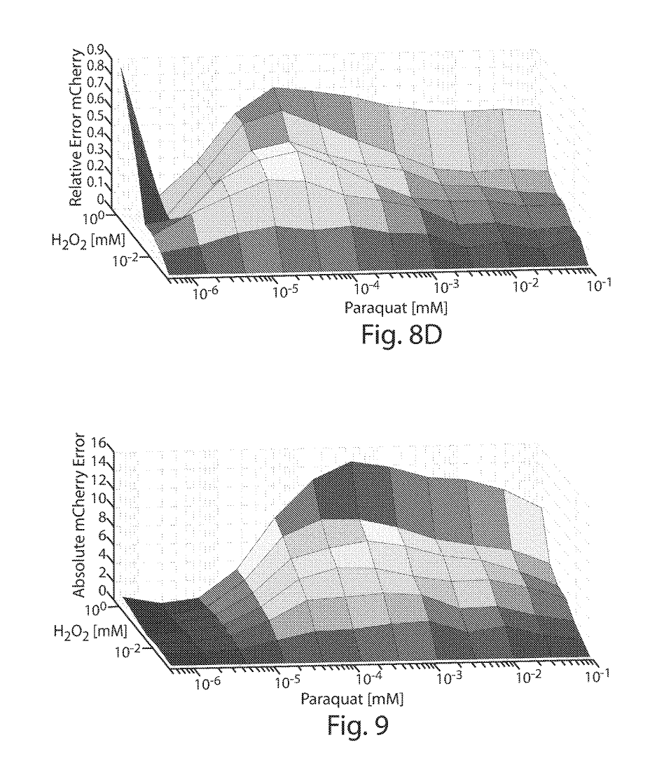

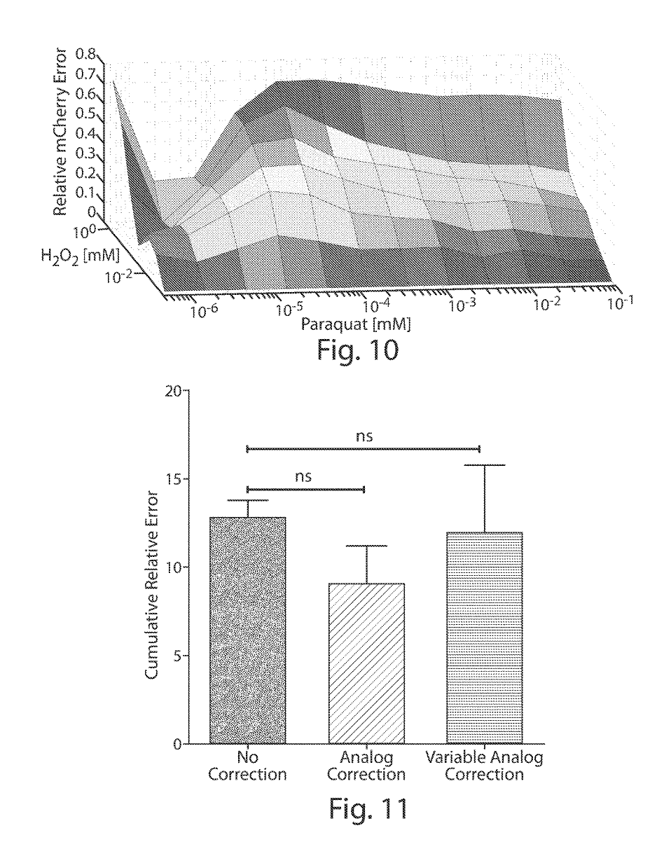

To quantify the amount of crosstalk in each biosensing circuit, the gene expression at any given paraquat and H₂O₂ concentration was calculated and compared to the gene expression at the same concentration of the gene circuit's target ROS in the absence of the non-target ROS (absolute error). The absolute error was normalized to gene expression in the absence of the non-target ROS (relative error) and these values were summed to get the total relative error (FIG. 8). The total relative error was 23.54 for the paraquat-sensing circuit (FIGS. 3H and 10) and 12.27 for the H₂O₂-sensing circuit (FIG. 11).

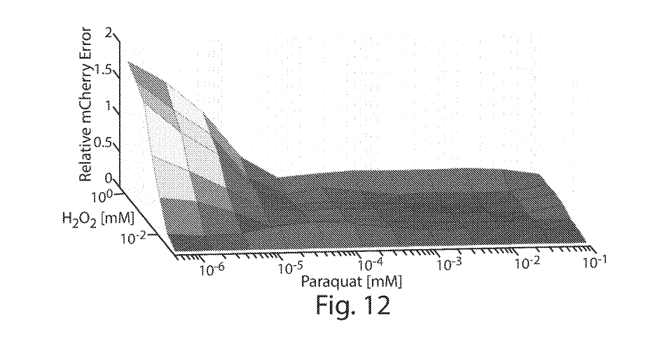

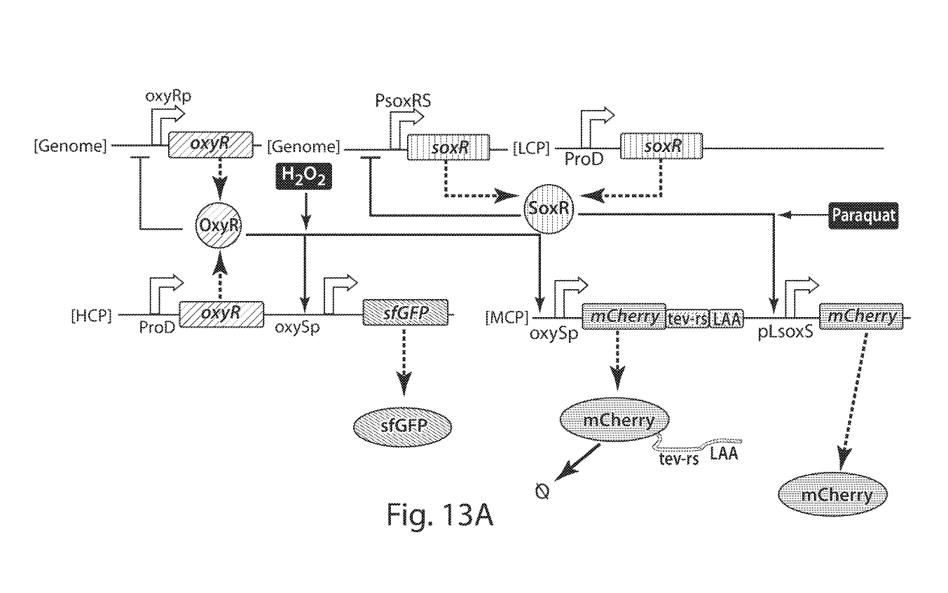

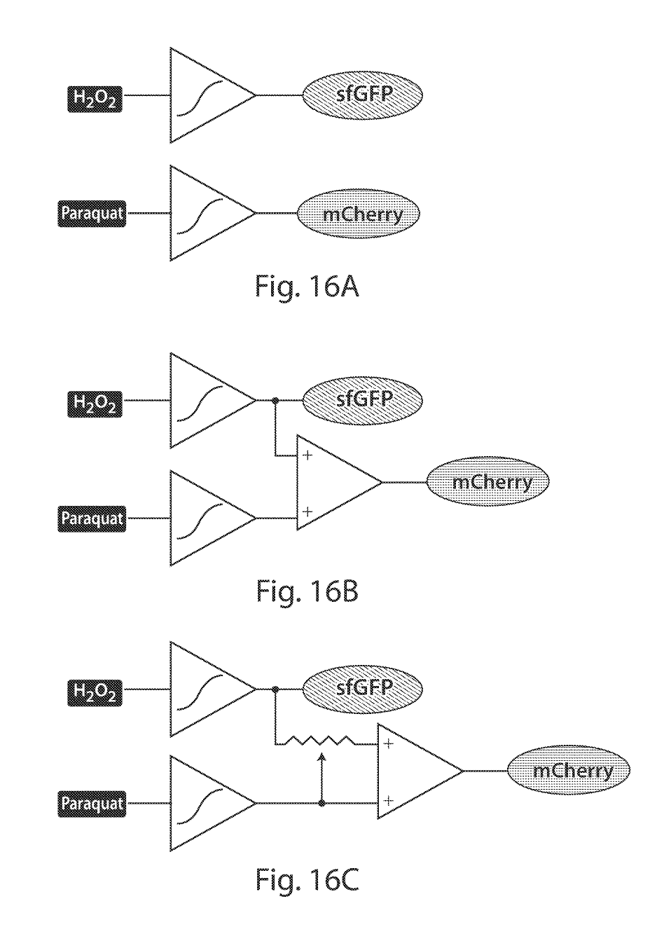

To address the crosstalk between H₂O₂ and the paraquat-sensing circuit, a synthetic circuit that introduced compensatory crosstalk was designed. The absolute error plot for the paraquat-sensing circuit (FIG. 9), which shows how observed gene expression deviates from gene expression at zero H₂O₂, indicated that the H₂O₂ crosstalk could be corrected by a circuit with a positive slope H₂O₂-to-mCherry function that is only activated at high paraquat when the paraquat sensor is also activated. Accordingly, an “analog correction circuit” was built by adding a second copy of mCherry under the control of an oxySp promoter to the dual-ROS sensing circuit (FIG. 3D). This circuit sums the mCherry flux from the oxySp and pLsoxS promoters (FIG. 16B). The analog correction circuit slightly overcorrected the H₂O₂ crosstalk at high paraquat concentrations and increased crosstalk at low paraquat concentrations (FIG. 12). Overall, the total relative error of the paraquat-sensing circuit was reduced to 21.21 (FIG. 3H) without significantly affecting the error of the H₂O₂-sensing circuit (sup FIG. 7).

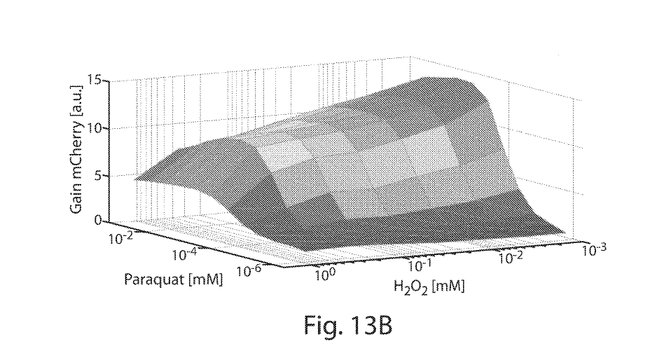

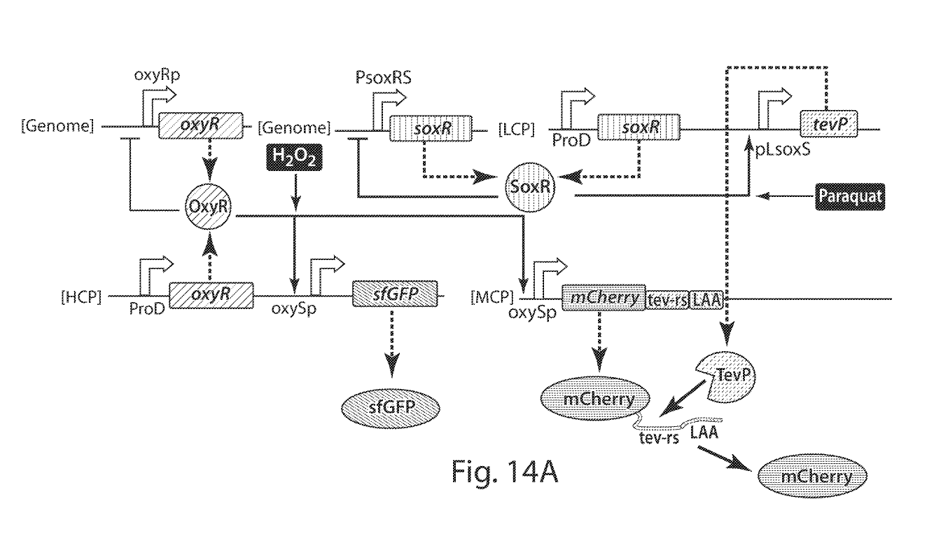

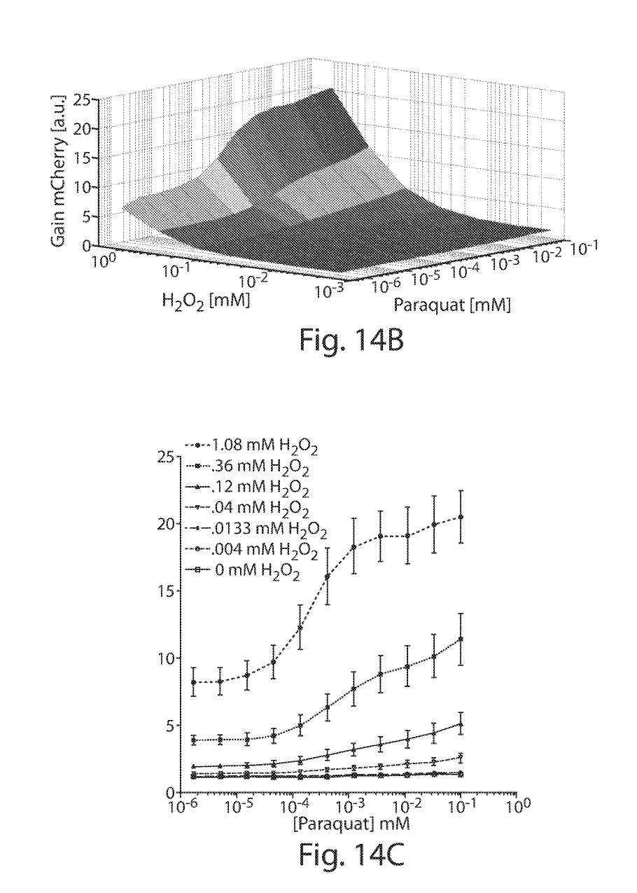

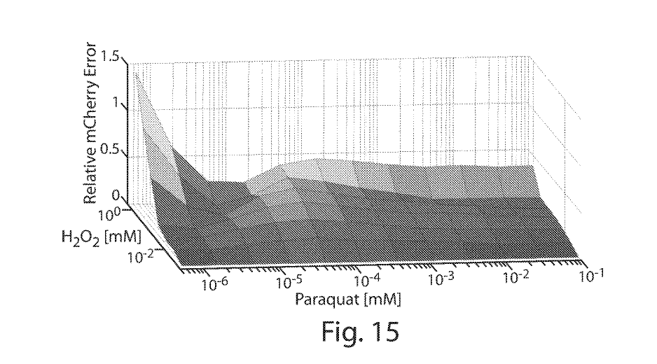

To address the increased crosstalk at low paraquat concentrations, a paraquat control of the analog corrective circuit was added to create a “variable analog correction circuit” (FIG. 3F, FIG. 16C). To do so, post-translational regulation rather than a transcriptional cascade was utilized to ensure that the circuit can compute within 1 hour of input stimulation. The C-terminus of the oxySp-controlled mCherry was fused to a TEV protease recognition sequence (TEV-rs) and an LAA degradation tag (FIG. 13). The mCherry protein is post-translationally stabilized when the LAA tag is cleaved off by TEV protease (TevP). The gene for tevP was placed under the control of pLsoxS. Thus, mCherry expressed from oxySp is unstable unless paraquat induces expression of tevP (FIG. 13). Indeed, the paraquat concentration controlled the magnitude of the mCherry output from oxySp (FIG. 14C). The oxySp-mCherry transfer function of the variable analog correction circuit (FIG. 14B) was similar to the absolute error curve for the initial dual-ROS sensing strain (FIG. 8). The variable analog correction circuit considerably reduced crosstalk at low paraquat concentrations while maintaining its corrective ability at high paraquat concentrations (FIG. 3G). The total relative error was 23.5 for the paraquat-sensing circuit (FIGS. 3H, 15) and 12.3 for the H₂O₂-sensing circuit (FIG. 11).

### Example 4

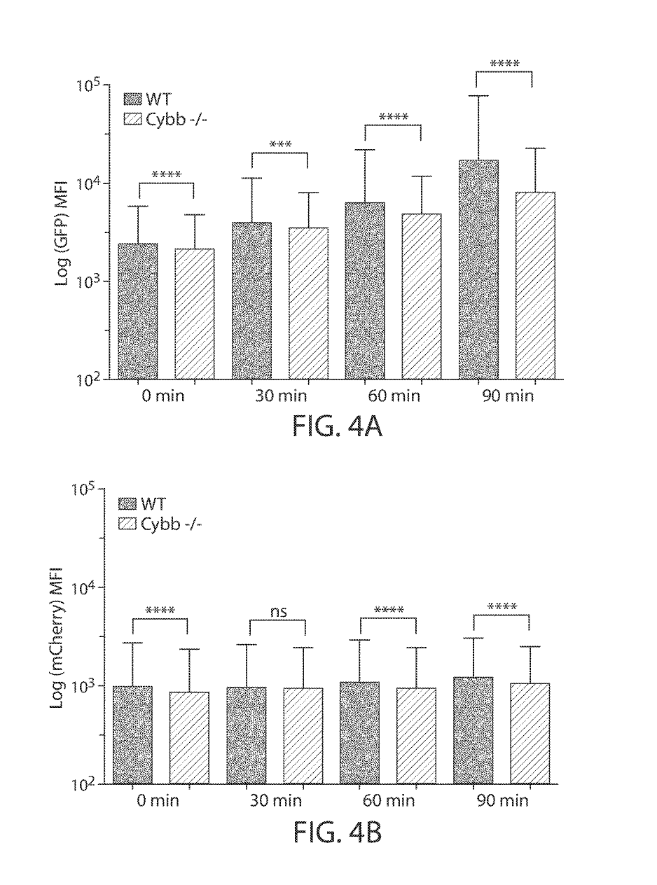

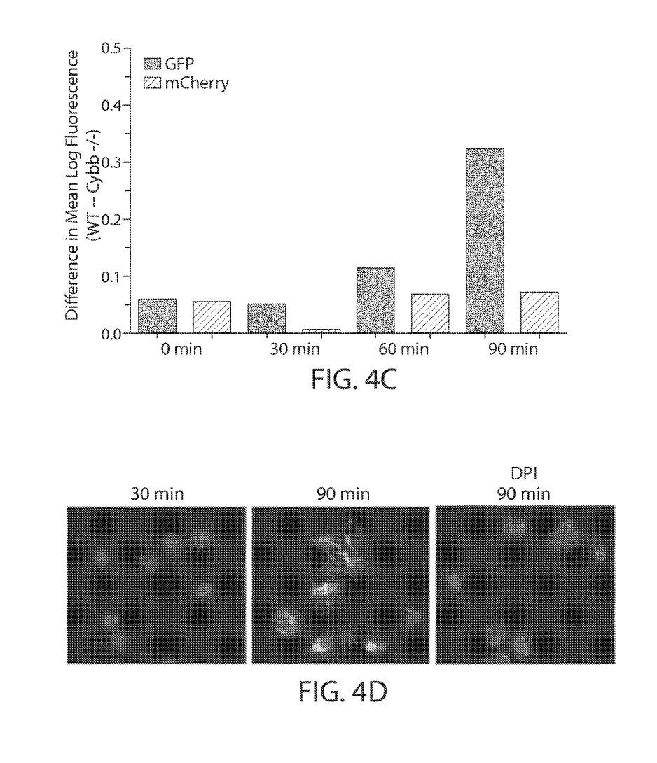

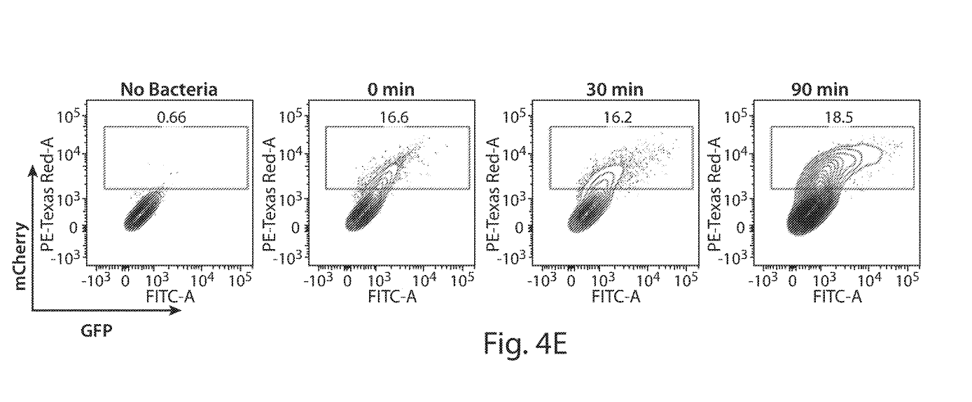

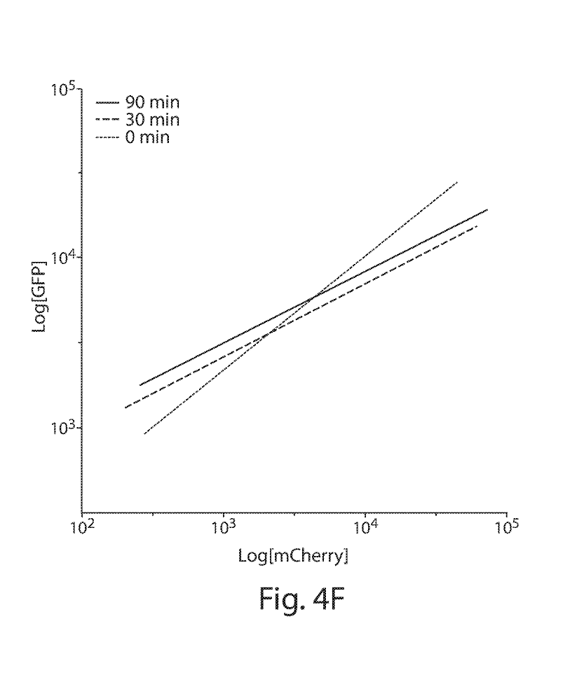

The ROS biosensor circuits can interface with mammalian immune cells and are capable of distinguishing between wild-type cells and those with a knockout in a gene linked to inflammatory bowel disease (IBD) (FIG. 4). The dual-ROS sensing strain was incubated with ex vivo murine bone-marrow-derived dendritic cells (BMDCs) from C57BL/6 (WT) or Cybb^{-/-} mice at a 1:1 ratio in mammalian culture media. The cells were chased at 30 minute time points up to 90 minutes, analyzed by fluorescence-activated cell sorting (FACS), and gated for live BMDCs. The H₂O₂-sensing circuit was capable of differentiating the different BMDC cell types at every time point tested (FIG. 4A), while the O₂⁻-sensing circuit (which sensed paraquat in vitro) was also able to do this at all time points except for at 30 minutes (FIG. 4B). The difference in mean fluorescence between the wild-type (WT) and Cybb−/− cells was greater for the H₂O₂ sensing circuit than for the O₂⁻-sensing circuit at every time point (FIG. 4C), suggesting that the H₂O₂-sensing circuit is a better differentiator for IBD-related BMDC phagocytotic processes than the O₂⁻-sensing circuit.

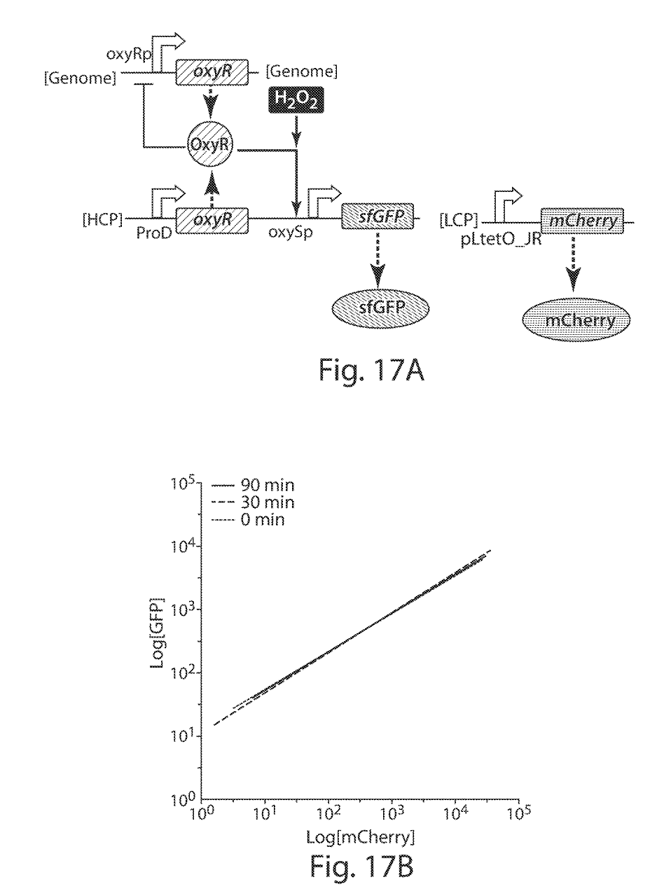

To determine whether the observed difference in GFP expression between cell types and across time points was a direct cause of BMDC-derived ROS, wild-type BMDCs were imaged with the dual-ROS E. coli. Fluorescent E. coli were localized to BMDCs and fluorescence was reduced by the Nox2 inhibitor diphenyleneiodonium (DPI), confirming the ROS-dependent activation of GFP expression (FIG. 4D). To study the relationship between fluorescence and the number of E. coli per BMDC, an E. coli strain with the H₂O₂-sensing circuit and constitutive mCherry expression was built (FIG. 17A). As expected, it was found that more E. coli are phagocytosed as time progresses (mCherry positive cells, (FIG. 4E)). A significant linear correlation was observed between BMDC GFP and mCherry fluorescence taken from all BMDC (FIG. 17B) or those that were phagocytotic (FIG. 4F). There does not appear to be a trend between the time point at which cells were analyzed and the slope of the mCherry-GFP relationship. This suggests that for the experiments in FIGS. 4A-4C, the difference in mean GFP fluorescence for a cell type between time points is a largely function of the number of E. coli per BMDC rather than increasing H₂O₂ per phagolysosome over time or the temporal dynamics of the sensor gene circuit.

The present disclosure provides analog biosensing circuits engineered based on quantitative performance metrics. Going forward, engineered organisms may increasingly utilize front-end analog sensors to enable complex computations based on environmental signals. For instance, a probiotic engineered to diagnose inflammation could process the analog signal from the H₂O₂-sensor with digital converters and memory units to enable a precise, noise-buffered, memorized measurement of H₂O₂ concentration in the mammalian gut. Diagnoses based on a front-end digital sensor would be less precise because such a sensor can only classify between two H₂O₂ concentrations (HI or LO). Most engineered organisms, however, will utilize multiple inputs to assess the environmental state. Thus, it is essential to characterize the analog crosstalk between input signal processing functions. The error metrics presented herein can be used to quantify such crosstalk and guide crosstalk correction.

The compensatory crosstalk correction method provided by the present disclosure is generalizable to other gene circuits. The crosstalk observed may arise, for example, from the metabolic connections between ROS or various interactions in E. coli's complex ROS gene regulatory network. Rather than trying to reduce this crosstalk by identifying and mutating the responsible interaction, the crosstalk was corrected by introducing additional, transcriptional crosstalk. This empirically demonstrates how the plasticity of gene regulatory networks can alter signal processing in living cells. Indeed, transcription factors evolve more rapidly than the genes they regulate and transcriptional networks are readily rewirable, with new connections often increasing cell fitness. Thus, natural gene networks may also implement the crosstalk correction method. Recognized gene-network motifs, such as feedforward loops, may serve to not only regulate the response to a specific input, but also serve to insulate the response from, or to interface the response with, other inputs. Such crosstalk correction motifs may be identified based on incongruities between transcriptional factor-DNA interactions and functional network behavior. FIG. 18 shows an example of what this may look like based on the ROS-sensing network is shown. The synthetic gene networks of the present disclosure, in some embodiments, use multiple operons to engineer signal integration because the rules governing the interaction of multiple transcription factors and polymerase at a single promoter, as found in natural gene networks, are not well understood.

### Materials and Methods for Examples 1-4

### Strains and Plasmids.

All plasmids were constructed with standard cloning procedures. Escherichia coli BW25113 (F-, DE(araD-araB)567, lacZ4787(del)::rrnB-3, LAM-, rph-1, DE(rhaD-rhaB)568, hsdR514) or Escherichia coli MG1655Pro (F-λ-ilvG-rfb-50 rph-1 laciQ tetR specR) were used for experiments as noted.

### Circuit Characterization.

Overnight cultures of E. coli were grown from glycerol freezer stocks, shaking aerobically at 37 degrees in LB medium with appropriate antibiotics: Carbenicillin (50 μg/ml), Kanamycin (30 μg/ml), and Spectinomycin (100 μg/ml). Overnight cultures were diluted 1:100 into fresh LB with antibiotics and grown 1.5 hours to an optical density at 600 nm between 0.2-0.4. The cell density was adjusted to 50,000 cells/μl and resuspended in Optimem Media+5% FBS (Invitrogen). The culture was transferred to a 96-well plate and inducers were added at appropriate concentrations via serial dilution. The inducers H₂O₂ and paraquat (methyl viologen dichloride hydrate) were purchased from Sigma Aldrich. The induced culture was grown for 1 hour shaking aerobically at 37 degrees. Cultures were then diluted 1:4 into a new 96-well plate containing 1×PBS and assayed on a BD LSRFortessa using the high-throughput sampler. At least 30,000 events were recorded for all circuit characterization experiments. GFP expression was measured via the FITC channel and mCherry expression was measured via the Texas Red channel. FCS files were exported and processed in FlowJo software. Events were gated for live E. coli via forward scatter area and side scatter area, and the geometric mean of the population was calculated.

### BMDC Experiments.

C57BL/6 control mice and Cybb−/− mice were obtained from Jackson Laboratories. Murine bone marrow derived dendritic cells (BMDCs) were then prepared from the murine subjects. In brief, bone marrow was harvested from long bones and cultured in GM-CSF for 6 days. Cells were then harvested and replated to confluence in antibiotic-free media supplemented with IFN-gamma (10 ng/ml). After 18 hours in culture, BMDCs were washed in PBS prior to addition of bacteria. E. coli was grown to an OD600 of 0.2-0.3, adjusted 50,000 cells/μl and resuspended in PBS. Bacteria were then added at a 1:1 volume ratio to BMDCs for 30 min at 37 degrees C. Plates were washed twice in PBS, resuspended in antibiotic-free media and chased in culture for the indicated time points. Subsequently, cells were harvested by gentle scraping and analyzed by FACS (BD LSRFortessa or C6 Accuri). Analysis was performed with FlowJo software.

### Calculating Output Fold-Change, Relative Input Range, Sensitivity and Utility

Best Fit Hill Function from Raw Data Calculation (FIG. 5A):

Hill functions are of the form:

$$y = \frac{bmax * {x}^{n}}{{k}_{d}^{n} + {x}^{n}} + C$$

Where C is fixed as the empirical Geometric Mean (y) at 0 input (x=0), and n, kd, and bmax are fit to the data.

### Output Fold-Induction (G) Calculation:

$$G = \frac{( bmax + C )}{C}$$

If the observed maximum gene expression is less than the theoretical bmax, then the observed maximum gene expression (observed bmax) rather than theoretical bmax is used to calculate output fold-induction. In this case, the out-fold induction is:

$$G = \frac{( observed bmax )}{C}$$

### Input Dynamic Range Calculation (FIG. 5B):

In the case where the theoretical bmax is less than the max gene expression observed

90% of maximum output (Y₉₀) is calculated as:

Y₉₀=C+0.9*bmax

10% of maximum output (Y₁₀) is calculated as:

Y₁₀=C+0.1*bmax

In the case where the theoretical bmax is greater than the max gene expression observed 90% of maximum output (Y₉₀) is calculated as:

Y₉₀=C+0.9*(observed bmax−C)

10% of maximum output (Y₁₀) is calculated as

Y₁₀=C+0.1*(observed bmax−C)

The Y₉₀ and Y₁₀ are interpolated to the X-axis to determine the X₉₀ and X₁₀, which define the input dynamic range.

The relative input range is calculated as

$$Relative Input Range = \frac{{X}_{90}}{{X}_{10}}$$

### Sensitivity Calculation (FIG. 5C):

Sensitivity is calculated using the Hill equation from above (with theoretical Bmax)

S(x)=(δy/y)/(δx/x)

### Utility Calculation (FIG. 5D):

Numerically integrate the sensitivity function over input values (x) relative to X₁₀ and multiply by the output fold-induction (G):

$$Utility = G * {\int }_{\frac{{X}_{10}}{{X}_{10}}}^{\frac{{X}_{90}}{{X}_{10}}} S ( x ) \partial ( \frac{x}{{X}_{10}} ) = \frac{G}{{X}_{10}} {\int }_{{X}_{10}}^{{X}_{90}} S ( x ) \partial x$$

### Calculating Cross-Talk Error

### Raw Cross-Talk Error Calculation (FIGS. 8A and 8B):

Raw cross-talk error is calculated by subtracting the gene expression at a given concentration of paraquat and H₂O₂ from gene expression at the same concentration of either paraquat or H₂O₂ and zero H₂O₂ or paraquat, respectively.

$$Raw cross \text{-} talk error = {GeneExpression}_{{parquat}_{a} , {H}_{2} {O}_{2}} \text{?} - {GeneExpression}_{{parquat}_{a} , {H}_{2} {O}_{2}} \text{?}$$

$$\text{?} \text{indicates text missing or illegible when filed}$$

### Absolute Cross-Talk Error Calculation (FIG. 8C):

Absolute cross-talk error is calculated by taking the absolute value of the raw cross-talk error. The absolute cross-talk error for each experimental replicate is averaged to get the shown plots of absolute cross-talk error.

$$Absolute cross \text{-} talk error = \vert {GeneExpression}_{{parquat}_{a} , {H}_{2} {O}_{2 b}} - {GeneExpression}_{{parquat}_{a} , {H}_{2} {O}_{20}} \vert$$

### Relative Cross-Talk Error Calculation (FIG. 8D):

Relative cross-talk error is calculated by adjusting the absolute raw cross-talk error to gene expression at a given concentration of either paraquat or H₂O₂ and zero H₂O₂ or paraquat, respectively. The relative cross-talk error for each experimental replicate is averaged to get the shown plots of relative cross-talk error.

$$Relative cross \text{-} talk error = \frac{\vert {GeneExpression}_{{parquat}_{a} , {H}_{2} {O}_{2 b}} - {GeneExpression}_{{parquat}_{a} , {H}_{2} {O}_{20}} \vert}{{GeneExpression}_{{parquat}_{a} , {H}_{2} {O}_{20 }}}$$

### Total Relative Cross-Talk Error Calculation:

To calculate the total relative cross-talk error, the relative cross-talk error at every concentration of paraquat and H₂O₂ is summed. The total relative cross-talk error for each experiment replicate is calculated independently and averaged to get the reported total relative cross-talk error.

$$Total relative cross \text{-} talk error = {\sum }_{{parquat}_{0} , {H}_{2} {O}_{20}}^{{parquat}_{max} , {H}_{2} {O}_{2 min }} \frac{\vert \begin{matrix}{GeneExpression}_{{parquat}_{a} , {H}_{2} {O}_{2 b}} - \\ {GeneExpression}_{{parquat}_{a} , {H}_{2} {O}_{20}}\end{matrix} \vert}{{GeneExpression}_{{parquat}_{a} , {H}_{2} {O}_{20}}}$$

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A biosensing circuit comprising:
(a) a first promoter responsive to a first input signal and operably linked to a nucleic acid encoding a first output molecule; and
(b) a second promoter responsive to the first input signal and operably linked to a nucleic acid encoding a copy of the first output molecule,
wherein the response of the second promoter to the first input signal is opposite the response of the first promoter to the first input signal such that the first input signal does not affect relative production of the first output molecule.

2. The biosensing circuit of claim 1, wherein the first promoter is responsive to a first input signal and a second input signal.

3. The biosensing circuit of claim 1 or claim 2, wherein (a) and (b) are on the same vector.

4. The biosensing circuit of any one of claims 1-3, wherein production of the first output molecule of (a) is decreased as a result of the first promoter responding to the first input signal.

5. The biosensing circuit of any one of claims 1-4, wherein production of the copy of the first output molecule of (b) is increased as a result of the second promoter responding to the first input signal.

6. The biosensing circuit of any one of claims 2-5, wherein production of the first output molecule of (a) is increased as a result of the first promoter responding to the second input signal.

7. The biosensing circuit of any one of claims 1-6, further comprising a third promoter responsive to the first input signal and operably linked to a nucleic acid encoding a second output molecule that is different from the first output molecule.

8. The biosensing circuit of any one of claims 1-7, further comprising a fourth promoter operably linked to a nucleic acid encoding a first biomolecule that binds to and regulates the first promoter and is responsive to the second input signal.

9. The biosensing circuit of claim 8, wherein activity of the first biomolecule is induced by the second input signal.

10. The biosensing circuit of any one of claims 1-9, further comprising a fifth promoter operably linked to a nucleic acid encoding a second biomolecule that binds to and regulates the second promoter and is responsive to the first input signal.

11. The biosensing circuit of claim 10, wherein activity of the second biomolecule is induced by the first input signal.

12. The biosensing circuit of any one of claims 1-11, wherein the copy of the first output molecule of (b) is fused to a protease recognition sequence.

13. The biosensing circuit of claim 12, wherein the protease recognition sequence is fused to a degradation tag.

14. The biosensing circuit of claim 12 or 13, further comprising a sixth promoter responsive to the second input signal and operably linked to a nucleic acid encoding a protease that cleaves the protease recognition sequence.

15. The biosensing circuit of any one of claims 2-14, wherein the second input signal is paraquat.

16. The biosensing circuit of claim 15, wherein the first promoter is a pLsoxS promoter.

17. The biosensing circuit of any one of claims 1-16, wherein the first input signal is peroxide.

18. The biosensing circuit of claim 17, wherein the second promoter is an oxySp promoter.

19. The biosensing circuit of any one of claims 8-18, wherein the first biomolecule is SoxR.

20. The biosensing circuit of any one of claims 10-19, wherein the second biomolecule is OxyR.

21. The biosensing circuit of any one of claims 14-20, wherein the protease is TevP.

22. A cell comprising the biosensing circuit of any one of claims 1-7.

23. A cell comprising the biosensing circuit of any one of claims 8-21.

24. The cell of claim 23, wherein the cell endogenously expresses the first and/or second biomolecule.

25. The cell of any one of claims 22-24, wherein the cell further comprises the first and/or second input signal.

26. The cell of any one of claims 22-25, wherein the cell is a bacterial cell.

27. A method of correcting crosstalk in a cell, comprising introducing into a cell, the biosensing circuit of any one of claims 1-21.

28. The method of claim 27, wherein the cell is a bacterial cell.
